(19) United States
(12) Patent Application Publication
(10) Pub. No.: US 2026/0260641 A1
(43) Pub. Date: Sep. 3, 2026

(54) METASTRUCTURE FOR IMPEDANCE MATCHING FOR MULTILAYER OR NON-UNIFORM BARRIER, ULTRASONIC PROBE COMPRISING SAME, AND ULTRASONIC IMAGE DIAGNOSIS APPARATUS COMPRISING SAME

(71) Applicants: KOREA INSTITUTE OF MACHINERY & MATERIALS, Daejeon (KR); CENTER FOR ADVANCED META-MATERIALS, Daejeon (KR)

(72) Inventors: Minwoo KWEUN, Daejeon (KR); Jae-Hyun KIM, Daejeon (KR); Hak-Joo LEE, Daejeon (KR); Hae Jin CHOI, Daejeon (KR); Jaehwa LEE, Daejeon (KR); Chun Guang PIAO, Daejeon (KR); Bongkyun JANG, Daejeon (KR); Kwangseop KIM, Daejeon (KR)

(21) Appl. No.: 18/875,293

(22) PCT Filed: Mar. 8, 2024
(86) PCT No.: PCT/KR2024/003013

§ 371 Date: Dec. 16, 2024

(30) Foreign Application Priority Data
Mar. 9, 2023 (KR) 10-2023-0031443
Mar. 9, 2023 (KR) 10-2023-0031444
Nov. 23, 2023 (KR) 10-2023-0164912
Nov. 23, 2023 (KR) 10-2023-0164913

(51) Int. Cl. G10K 11/02; B06B 1/06

(52) U.S. Cl. CPC G10K 11/02; B06B 1/067

(57) ABSTRACT

In a metastructure for impedance matching for multilayer or non-uniform barrier, an ultrasonic probe having same, and an ultrasonic image diagnosis apparatus having same, the metastructure is arranged to be spaced apart from a surface of a multilayer barrier by a predetermined matching distance toward an ultrasonic transducer, in an intermediate material provided between the ultrasonic transducer and the multilayer barrier. The metastructure is configured to minimize a difference between a surface impedance at the matching distance and a characteristic impedance of the intermediate material.

### BACKGROUND OF THE INVENTION

### Technical Field

Exemplary embodiments of the present invention relate to a metastructure for impedance matching for multilayer or non-uniform barrier, an ultrasonic probe having same, and an ultrasonic image diagnosis apparatus having same. More particularly, exemplary embodiments of the present invention relate to a metastructure for impedance matching for multilayer or non-uniform barrier, an ultrasonic probe having same, and an ultrasonic image diagnosis apparatus having same, for imaging the detection target area that exists beyond a multilayer barrier or non-uniform barrier with different impedance properties, such as the skull.

### Discussion of the Related Art

Metamaterials use periodic artificial structures to implement wave characteristics that are difficult for natural materials to possess, such as zero refractive index and negative refractive index. Research on metamaterials, which was active in the electromagnetic field in the past, has recently moved rapidly to the acoustics field, and research on acoustic metamaterials using the wave characteristics of acoustic waves is becoming more active.

For example, acoustic metamaterials can be used to locally focus sound in an area smaller than the wavelength, freely change the path of sound, or improve the image quality of sound wave and ultrasonic imaging. To achieve this, artificial structure design technology that freely adjusts elasticity, density, and refractive index through periodic arrangement of structures smaller than the wavelength is essential.

Recently, research has been conducted on so-called metasurfaces, metamaterial layers, and metastructures, which are created by arranging unit structures designed based on physical principles on a two-dimensional thin film.

Meanwhile, the skull, the bone tissue that protects the brain, is a connective tissue hardened by calcium and has a multilayered structure with cortical bone stacked on the inside and outside with cancellous bone in between. Cortical bone is a relatively dense hard tissue, and cancellous bone is a relatively sparse, porous soft tissue. In other words, cortical bone and cancellous bone have different physical properties as well as thickness, so the skull forms a multilayer barrier when ultrasonic waves penetrate.

In this way, the skull, which is made up of multilayer barrier structures such as cortical bone and cancellous bone, has high reflectance and low transmittance due to impedance differences at the interface of each barrier structure during medical ultrasonic imaging using ultrasonic waves or treatment using focused ultrasonic waves.

In addition, existing metasurfaces, metamaterial layers, and metastructures basically perform impedance matching on the interface of a single barrier through matching of ¼ wavelength (λ). In the case of a multilayer barrier, such as a skull with cortical bone and cancellous bone, there are many difficulties in designing impedance matching with high transmittance because all multiple effective impedances of the interface of the multilayer barrier must be considered.

In addition, since the thickness and physical properties of each layer constituting the cancellous bone and cortical bone in the skull are different for each individual, matching individual impedance is more difficult.

On the other hand, an ultrasonic imaging device is a device that converts electrical signals into mechanical vibrations using an ultrasonic transducer to transmit ultrasonic waves, and to receive signals reflected from an object, convert them into electrical signals, process them, and convert them into images.

The ultrasonic probe used in a conventional ultrasonic imaging device basically has a lens, a front material, a piezoelectric element (ultrasonic transducer), and a back material. Since a front material has a role in matching the impedance difference between the ultrasonic transducer and the soft tissue of the human body, it is difficult to transmit and receive ultrasonic signals by penetrating barriers that have a large impedance difference from human soft tissue, such as bone or gas (air layer existing inside the digestive system).

In addition, even if the impedance of the front material is matched considering the bones of the human body using an existing matching technique (impedance matching layer with a thickness of ¼ wavelength (λ)), it is difficult for the ultrasonic waves generated from the probe to first penetrate soft tissue such as human skin. Accordingly, it is difficult to apply the conventional ultrasonic probe front material technology to obtain images of soft tissues inside the human body by penetrating bone.

Related prior arts include Koran patent No. 10-2093248.

### SUMMARY

Exemplary embodiments of the present invention provide a metastructure for impedance matching for multilayer, capable of transmittance by calculating the effective impedance of a multilayer barrier such as the skull as the surface impedance regardless of the number of layers or interfaces of the barrier, rather than expressing it as the characteristic impedance of each barrier layer.

In addition, exemplary embodiments of the present invention also provide a metastructure for impedance matching for multilayer with anti-resonant vibration mode that may implement impedance matching for multilayer barriers.

In addition, exemplary embodiments of the present invention also provide a metastructure for impedance matching for non-uniform barrier capable of implementing impedance matching for non-uniform barriers with different physical properties not only in the direction of sound wave transmission but also in the direction crossing the direction of ultrasonic transmission.

In addition, exemplary embodiments of the present invention also provide a metastructure for impedance matching for non-uniform barrier capable of maximizing overall ultrasonic transmittance by performing regional impedance matching for non-uniform barriers.

In addition, exemplary embodiments of the present invention also provide a metastructure for impedance matching for multilayer or non-uniform barrier capable of setting design variables such as thickness and matching distance based on the intensity of reflected or transmitted ultrasonic wave.

In addition, exemplary embodiments of the present invention also provide an ultrasonic probe having the metastructure for impedance matching for multilayer or non-uniform barrier.

In addition, exemplary embodiments of the present invention also provide an ultrasonic image diagnosis apparatus having the metastructure for impedance matching for multilayer or non-uniform barrier.

According to one aspect of the present invention, the metastructure is arranged to be spaced apart from a surface of a multilayer barrier by a predetermined matching distance toward an ultrasonic transducer, in an intermediate material provided between the ultrasonic transducer and the multilayer barrier. The metastructure is configured to minimize a difference between a surface impedance at the matching distance and a characteristic impedance of the intermediate material.

In an exemplary embodiment, the metastructure may have an anti-resonance mode in which magnitudes of positive and negative vibrations in a natural vibration mode are substantially equal.

In an exemplary embodiment, the metastructure may include a first material layer having a first elastic modulus and a first density, and a second material layer having a second elastic modulus and a second density. The first and second material layers may be stacked in an ultrasonic transmission direction. The first and second moduli may be different from each other, or the first and second density may be different from each other.

In an exemplary embodiment, the second material layer may be disposed on front and back sides of the first material layer with respect to the ultrasonic transmission direction.

In an exemplary embodiment, the second material layer may include a pattern unit having first and second areas, the first area may expose the first material layer to outside, and the second area may cover the first material layer without exposing the first material layer.

In an exemplary embodiment, the metastructure may be a plural, and the plurality of the metastructures may be arranged to tile each other in a direction crossing an ultrasonic transmission direction.

In an exemplary embodiment, the metastructure may include a first metastructure spaced apart by a first matching distance from the surface of the multilayer barrier, and a second metastructure space apart by a second matching distance different from the first matching distance from the surface of the multilayer barrier.

According to another aspect of the present invention, a metastructure is arranged to be spaced apart from a surface of a multilayer barrier by a predetermined matching distance toward an ultrasonic transducer, in an intermediate material provided between the ultrasonic transducer and the multilayer barrier. The metastructure is configured to minimize a reflection coefficient of a system transfer matrix which consisting of a product of a transfer matrix expressed as a characteristic impedance and a phase change of each of the metastructure disposed at the matching distance, the intermediate material and the multilayer barrier.

In an exemplary embodiment, the reflection coefficient of a system transfer matrix may be minimized by the matching distance or a thickness of the metastructure.

In an exemplary embodiment, the metastructure may have an impedance twice or more than an impedance of the intermediate material or an impedance substantially the same as the characteristic impedance of the multilayer barrier, to expand a range of change in the system transfer matrix by intensifying multiple internal reflections of an ultrasonic wave propagating between the metastructure and the multilayer barrier.

According to still another aspect of the present invention, a metastructure is disposed within an intermediate material provided between an ultrasonic transducer and a barrier. The metastructure includes first and second metastructures. The first metastructure is spaced apart by a first matching distance from a first surface area of the barrier toward the transducer, and is configured to minimize a difference between a first surface impedance at the first matching distance and a characteristic impedance of the intermediate material. The second metastructure is spaced apart by a second matching distance from a second surface area of the barrier adjacent to the first surface area toward the transducer, and is configured to minimize a difference between a second surface impedance at the second matching distance and a characteristic impedance of the intermediate material.

In an exemplary embodiment, the first metastructure may have a first effective radiation area corresponding to the first surface area and the second metastructure may have a second effective radiation area corresponding to the second surface area. Each of the first and second effective radiation areas may have a width smaller than ½ wavelength (λ). According to still another aspect of the present invention, the metastructure is disposed within an intermediate material provided between an ultrasonic transducer and a barrier. The metastructure includes first and second metastructures. The first metastructure is spaced apart by a first matching distance from a first surface area of the barrier toward the transducer, and is configured to minimize a reflection coefficient of a first system transfer matrix which consisting of a product of a first transfer matrix expressed as a characteristic impedance and a phase change of each of the first metastructure disposed at the first matching distance, the intermediate material and the barrier. The second metastructure is spaced apart by a second matching distance from a second surface area of the barrier adjacent to the first surface area toward the transducer, and is configured to minimize a reflection coefficient of a second system transfer matrix which consisting of a product of a second transfer matrix expressed as a characteristic impedance and a phase change of each of the second metastructure disposed at the second matching distance, the intermediate material and the barrier.

In an exemplary embodiment, the metastructure may include a 1-1 metastructure disposed from the first surface area by a 1-1 matching distance, and a 1-2 metastructure disposed from the first surface area by a 1-2 matching distance different from the 1-1 matching distance.

In an exemplary embodiment, the first metastructure may have an impedance twice or more than an impedance of the intermediate material or an impedance substantially the same as the characteristic impedance of the barrier, to expand a range of change in the first system transfer matrix by intensifying multiple internal reflections of an ultrasonic wave propagating between the first metastructure and the barrier.

According to still another aspect of the present invention, the ultrasonic probe includes the metastructure and an ultrasonic transducer configured to provide an ultrasonic wave to the metastructure.

In an exemplary embodiment, the metastructure may move in an ultrasonic transmission direction with respect to the ultrasonic transducer.

According to still another aspect of the present invention, the ultrasonic probe includes the metastructure and an ultrasonic transducer configured to provide an ultrasonic wave to the metastructure.

In an exemplary embodiment, the first metastructure or the second metastructure may move in an ultrasonic transmission direction with respect to the ultrasonic transducer.

According to still another aspect of the present invention, the ultrasonic image diagnostic apparatus includes the metastructure, an ultrasonic transducer, a signal processing unit and a metastructure control unit. The ultrasonic transducer is configured to provide an ultrasonic wave to the metastructure. The signal processing unit is configured to process a signal received in the ultrasonic transducer, to generate an ultrasonic image. The metastructure control unit is configured to control at least one of physical properties, thickness and matching distance of the metastructure, based on the ultrasonic image generated in the signal processing unit.

According to some exemplary embodiments of the present invention, the ultrasonic wave may be substantially 100% transparent across multilayer or non-uniform barriers.

In addition, when adjusting the matching distance of the metastructure or adjusting the physical properties such as the elastic modulus and pattern portion of the metastructure, the ultrasonic wave may be effectively transmitted while actively coping with the environmental conditions of the multilayer barrier.

In addition, the ultrasonic wave may be transmitted effectively despite individual differences in the physical properties of the skull as well as differences in physical properties by location.

In addition, the metastructure may be widely used. For example, the metastructure may be applied to medical ultrasonic imaging diagnosis and treatment in which the ultrasonic waves are transmitted into multilayered tissues containing substances with impedance differences such as bone tissue, soft tissue, gas, etc. In addition, the metastructure may be applied to various non-destructive testing industries that use the ultrasonic waves to penetrate multilayer physical barriers containing solid (metal, ceramic, plastic) or fluid materials (composite materials, insulation materials, rust or sludge in pipes, etc.).

### BRIEF DESCRIPTION OF THE DRAWINGS

FIG. 1 is a conceptual diagram for explaining the surface impedance placed in the intermediate material provided between the ultrasonic transducer and the multilayer barrier when the ultrasonic wave is incident from the ultrasonic transducer toward the multilayer barrier, an ultrasonic probe in which a metastructure according to an example embodiment of the present invention is applied;

FIG. 2 is a schematic diagram for explaining the positioning process of the surface impedance placed on the water as a medium when the ultrasonic wave from the ultrasonic transducer is incident on the water as a medium and the skull as the multilayer barrier, in the ultrasonic probe of FIG. 1;

FIG. 3 is a schematic diagram illustrating the impedance matching of a metamaterial with an effective area density placed on water, a medium material, when the ultrasonic wave from the ultrasonic transducer is incident towards water, a medium material, and the skull, which is the multilayer barrier, in the ultrasonic probe of FIG. 1;

FIG. 4A, FIG. 4B and FIG. 4C are side views and perspective views illustrating the metastructure of FIG. 1;

FIG. 5 is a graph showing transmittance by frequency when the metastructure of FIG. 4A is applied and when the metastructure of FIG. 4A is not applied;

FIG. 6 is a perspective view illustrating a metastructure according to another example embodiment of the present invention;

FIG. 7 is a schematic view illustrating a state to which a metastructure according to still another example embodiment of the present invention is applied;

FIG. 8A is a schematic view illustrating a state to which a metastructure according to still another example embodiment of the present invention is applied, and illustrating the metastructure for impedance matching for a barrier with a porous structure;

FIG. 8B is a schematic view showing the approximation of the porous structure within a multilayer barrier with a uniform material in FIG. 8A;

FIG. 9A is an image showing a result of simulation verification that ultrasonic transmission is improved when the metastructure for impedance matching is applied to the barrier having the porous structure of FIG. 8A;

FIG. 9B is a graph showing a change in pressure distribution along the right border of FIG. 9A due to the porous structure of the metastructure of FIG. 8A;

FIG. 10 is a schematic view illustrating a metastructure for impedance matching for multilayer for the multi barrier according to still another example embodiment of the present invention;

FIG. 11 is a graph showing wavelength characteristics of surface impedance at a location spaced apart by a matching distance from the surface of a non-uniform barrier whose physical properties continuously change along the direction intersecting the direction of ultrasonic transmission;

FIG. 12 is a schematic view illustrating a metastructure according to still another example embodiment of the present invention;

FIG. 13 is a schematic view illustrating a metastructure according to still another example embodiment of the present invention;

FIG. 14 is a graph showing impedance matching conditions for designing the A1 layer of FIG. 13;

FIG. 15 is a graph showing impedance matching conditions for designing the A2 layer of FIG. 13;

FIG. 16 is a simulated result showing transmittance before applying the metastructure;

FIG. 17 is a simulated result showing transmittance after applying the metastructure according to the example embodiments of the present invention;

FIG. 18 is a block diagram illustrating the conventional ultrasonic probe;

FIG. 19A is a block diagram illustrating an ultrasonic probe according to still another example embodiment of the present invention;

FIG. 19B is a block diagram illustrating an ultrasonic probe according to still another example embodiment of the present invention;

FIG. 19C is a block diagram illustrating an ultrasonic probe according to still another example embodiment of the present invention;

FIG. 20 is a block diagram for explaining a control of the metastructure, in an ultrasonic probe according to still another example embodiment of the present invention;

FIG. 21 is a block diagram illustrating the conventional ultrasonic image diagnosis apparatus;

FIG. 22 is a block diagram illustrating an ultrasonic image diagnosis apparatus according to still another example embodiment of the present invention;

FIG. 23 is a block diagram for explaining an operation method of the ultrasonic image diagnosis apparatus of FIG. 21;

FIG. 24 is a flow chart for explaining an ultrasonic image diagnosis method using the apparatus of FIG. 22; and

FIG. 25A and FIG. 25B are graphs showing an insertion position and reflectance change according to repetitive control of the metastructure, the method of FIG. 24.

<Reference numerals>

1: ultrasonic transducer
2: barrier
3: intermediate material
10, 10′, 20, 30, 50: metastructure
11: first metastructure
12: second metastructure
40: porous multilayer barrier
41: outer layer (outer cortex) within multilayer barrier
42: porous structure within multilayer barrier
43: inner layer (outer cortex) within multilayer barrier
60, 61, 62: background medium
63: object
70: ultrasonic transducer
80: ultrasonic transmission signal
81: transmission direction ultrasonic wave
82: barrier penetrating ultrasonic wave
83: object reflecting signal
84: receiving direction ultrasonic wave
85: ultrasonic receiving signal
86: gap between metastructure and barrier
100: conventional ultrasonic probe
101, 411, 421: lens
102, 413: front material
103, 414, 423, 502: ultrasonic transducer (piezoelectric element)
104, 415, 424: rear material
110: ultrasonic transceiver
120: transmission pulse generator
130: analog digital converter
140: transmission/reception focus unit
150: intermediate signal processing unit
160: image processing unit
161: B-mode image acquisition
162: blood flow Doppler signal and image acquisition
300, 410, 420, 500: (meta) ultrasonic probe
301, 412, 422, 501: metastructure
350: metal structure control unit

### DETAILED DESCRIPTION OF THE INVENTION

The present invention is described more fully hereinafter with reference to the accompanying drawings, in which exemplary embodiments of the present invention are shown. The present invention may, however, be embodied in many different forms and should not be construed as limited to the exemplary embodiments set forth herein. Rather, these exemplary embodiments are provided so that this disclosure will be thorough and complete, and will fully convey the scope of the present invention to those skilled in the art.

It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, the present invention will be explained in detail with reference to the accompanying drawings.

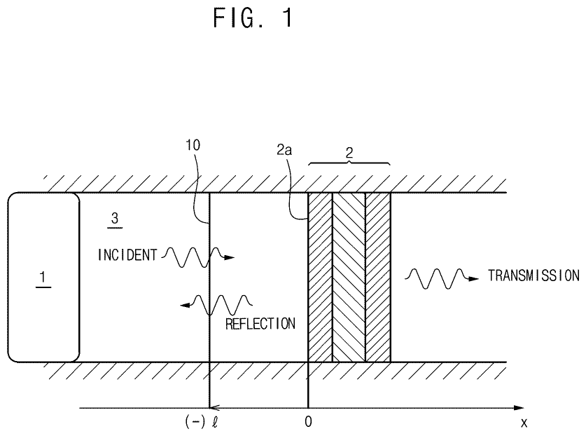

FIG. 1 is a conceptual diagram for explaining the surface impedance placed in the intermediate material provided between the ultrasonic transducer and the multilayer barrier when the ultrasonic wave is incident from the ultrasonic transducer toward the multilayer barrier, an ultrasonic probe in which a metastructure according to an example embodiment of the present invention is applied.

Referring to FIG. 1, the metastructure 10 according to the present example embodiment is used for an ultrasonic probe or an ultrasonic image diagnostic apparatus having the ultrasonic probe.

Here, the ultrasonic probe basically transmits ultrasonic waves into an interior of a multilayer barrier 2 having different physical properties and generates an ultrasonic image of a detection target area that exists beyond the multilayer barrier 2. The metastructure may basically be said to be an impedance matching metastructure that performs impedance matching on a barrier interface, when generating the ultrasonic image of the area beyond the barrier structure.

In general, impedance matching for the boundary surface of a single barrier is performed through matching of ¼ wavelength (λ). However, this impedance matching is difficult to apply in the case of a multilayer barrier such as skulls with cortical and cancellous bone. In other words, there are many difficulties in designing impedance matching that reaches a practical 100% transmittance because all of the multiple effective impedances of the interface of the multilayer barrier must be considered.

Thus, in the present example embodiment, instead of expressing the effective impedance of the multilayer barrier 2 as characteristic impedance for each layer of the barrier, the impedance matching method is calculated by calculating the surface impedance, regardless of the number of layers of the barrier 2, that is, the number of interfaces.

Referring to FIG. 1, the metastructure 10 according to the present example embodiment is arranged to be spaced apart from the surface 2a of the multilayer barrier 2 by a matching distance (toward the ultrasonic transducer 1, inside of an intermediate material 3 formed between the ultrasonic transducer 1 and the multilayer barrier 2.

Thus, the metastructure 10 according to the present example embodiment ensures that the imaginary part of the surface impedance at the matching distance (is zero so that the surface impedance at the predetermined matching distance (becomes the same as the characteristic impedance of the intermediate material 3, and thus the ultrasonic wave may be substantially 100% transparent across the multilayer barrier 2.

By allowing virtually 100% ultrasonic waves to pass through the multilayer barrier with different physical properties, it is possible to generate high-resolution images of the detection target area that exists beyond the multilayer barrier 2.

Surface impedance refers to the sound pressure on a surface divided by the particle speed when a plane sound wave is incident perpendicularly on a surface.

When the ultrasonic wave is incident from the ultrasonic transducer 1 (piezoelectric element) toward the multilayer barrier 2, the surface impedance (Zₗ) disposed within the intermediate material 3 may be basically expressed as <Equation 1> below.

$$\\ \begin{matrix}{Z}_{l} = \frac{p ( x = - l )}{u ( x = - l )} & < Equation 1 >\end{matrix}$$

At this time, the ultrasonic wave is incident substantially perpendicularly toward the multilayer barrier 2, x means the position in the ultrasonic transmission direction, and the position of the surface 2a of the multilayer barrier 2 is x=O.

In the end, p(x=−1) means the sound pressure at a position l away from the surface 2a of the multilayer barrier 2 toward the ultrasonic transducer 1, u(x=−1) means the particle velocity at a position l away from the surface 2a of the multilayer barrier 2 toward the ultrasonic transducer 1.

In this case, the intermediate material 3 may be water, or may be a material having a characteristic impedance similar to water.

Here, in the present example embodiment, since the surface impedance, such as the representative physical properties of the multilayer barrier 2, is considered, there is no need to introduce additional metamaterial layers that offset the unique characteristic impedance of each layer, as the number of layers of the multilayer barrier 2 increases, by directly considering the characteristic impedance of the barrier, like conventional complementary metamaterials.

It is natural that the surface impedance in the present example embodiment is related to the unique characteristic impedance of each layer of the multilayer barrier 2, but it may be calculated as one representative value without introducing additional metamaterial layers that offset the unique characteristic impedance of each layer.

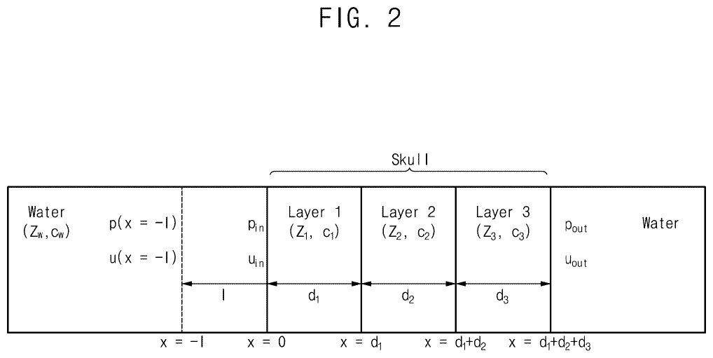

FIG. 2 is a schematic diagram for explaining the positioning process of the surface impedance placed on the water as a medium when the ultrasonic wave from the ultrasonic transducer is incident on the water as a medium and the skull as the multilayer barrier, in the ultrasonic probe of FIG. 1.

When the intermediate material (w: water), an outer cortical bone (Layer 1), a cancellous bone (Layer 2), and an inner cortical bone (Layer 3) are sequentially layered along the ultrasonic transmission direction, the surface impedance (Zₗ) may be calculated as one representative value as shown in <Equation 2> below.

$$\\ \begin{matrix}{Z}_{l} = \frac{( {C}_{w} {M}_{11} + {iZ}_{w} {S}_{w} {M}_{21} ) {Z}_{w} + {C}_{w} {M}_{12} + {iZ}_{w} {S}_{w} {M}_{22}}{( i \frac{{S}_{w}}{{Z}_{w}} {M}_{11} + {C}_{w} {M}_{21} ) {Z}_{w} + i \frac{{S}_{w}}{{Z}_{w}} {M}_{12} + {C}_{w} {M}_{22}} & < Equation 2 >\end{matrix}$$

$$\\ \begin{matrix}{M}_{11} = {C}_{1} {C}_{2} {C}_{3} - \frac{{Z}_{1}}{{Z}_{2}} {S}_{1} {S}_{2} {S}_{3} - \frac{{Z}_{1}}{{Z}_{3}} {S}_{1} {C}_{2} {S}_{3} - \frac{{Z}_{2}}{{Z}_{3}} {C}_{1} {S}_{2} {S}_{3} & < Equation 2 - 1 >\end{matrix}$$

$$\\ \begin{matrix}{M}_{12} = i ( {Z}_{1} {S}_{1} {C}_{2} {C}_{3} + {Z}_{2} {C}_{1} {S}_{2} {C}_{3} + {Z}_{3} {C}_{1} {C}_{2} {S}_{3} - \frac{{Z}_{1} {Z}_{3}}{{Z}_{2}} {S}_{1} {S}_{2} {S}_{3} ) & < Equation 2 - 2 >\end{matrix}$$

$$\\ \begin{matrix}{M}_{21} = i ( \frac{{S}_{1} {C}_{2} {C}_{3}}{{Z}_{1}} + \frac{{C}_{1} {S}_{2} {C}_{3}}{{Z}_{2}} + \frac{{C}_{1} {C}_{2} {S}_{3}}{{Z}_{3}} - \frac{{Z}_{2}}{{Z}_{1} {Z}_{3}} {S}_{1} {{S}_{}}_{2} {S}_{3} ) & < Equation 2 - 3 >\end{matrix}$$

$$\\ \begin{matrix}{M}_{22} = {C}_{1} {C}_{2} {C}_{3} - \frac{{Z}_{2}}{{Z}_{1}} {S}_{1} {S}_{2} {C}_{3} - \frac{{Z}_{3}}{{Z}_{1}} {S}_{1} {C}_{2} {S}_{3} - \frac{{Z}_{3}}{{Z}_{2}} {C}_{1} {S}_{2} {S}_{3} & < Equation 2 - 4 >\end{matrix}$$

$$\\ \begin{matrix}{C}_{w} = cos \frac{ω l}{{c}_{w}} & < Equation 2 - 5 >\end{matrix}$$

$$\\ \begin{matrix}{S}_{w} = sin \frac{ω l}{{c}_{w}} & < Equation 2 - 6 >\end{matrix}$$

$$\\ \begin{matrix}{C}_{j} = cos \frac{ω {d}_{j}}{{c}_{j}} & < Equation 2 - 7 >\end{matrix}$$

$$\\ \begin{matrix}{S}_{j} = sin \frac{ω {d}_{j}}{{c}_{j}} j = 1 , 2 , 3 & < Equation 2 - 8 >\end{matrix}$$

Here, Z_{w}=ρ_{w}C_{w} is the characteristic impedance of the water, which is the intermediate material, and Zⱼ=ρⱼcⱼ(j=1,2,3) is the characteristic impedance of the j-th barrier (the outer cortical bone/the cancellous bone/the inner cortical bone) layer.

In addition, ρ_{w} is a density of the water, ρⱼ(j=1,2,3) is a density of the j-th barrier.

In addition, c_{w} is a speed of sound in the waver, cⱼ(j=1,2,3) is a speed of sound in the j-th barrier.

In addition, in <Equation 2-1> to <Equation 2-4>, Mⱼₖ is a transfer matrix component. The transfer matrix component expresses the relationship between pₒᵤₜ and uₒᵤₜ, and between pᵢₙ and uᵢₙ. pₒᵤₜ and uₒᵤₜ are respectively an ultrasonic pressure field and a particle velocity field in the intermediate material (water) immediately after passing through the multilayer barrier (outer cortical bone/cancellous bone/inner cortical bone) layer. pᵢₙ and uᵢₙ are respectively an ultrasonic pressure field and a particle velocity field at the surface (x=O) of a multilayer barrier (outer cortical bone/cancellous bone/inner cortical bone) just before passing through it. Here, each transfer matrix (Mⱼₖ) component includes a mass density, an ultrasonic propagation speed, and a thickness (dⱼ) of each layer of the multilayer barrier (outer cortical bone/cancellous bone/inner cortical bone).

For reference, the intermediate material, water, and the outer cortical bone, the cancellous bone, and the inner cortical bone of the skull have the physical properties as shown in [Table 1] below.

TABLE 1

| | | outer | | inner |
|---|---|---|---|---|
| | | cortical | cancellous | cortical |
| Layer | water | bone | bone | bone |
| thickness [mm] | — | 2.0 | 2.5 | 1.5 |
| speed of sound [m/s] | 1480 | 3514.86 | 2117.5 | 3514.86 |
| density [kg/m3] | 997 | 1908 | 1178.3 | 1908 |
| impedance [Mrayl] | 1.48 | 6.7 | 2.5 | 6.7 |

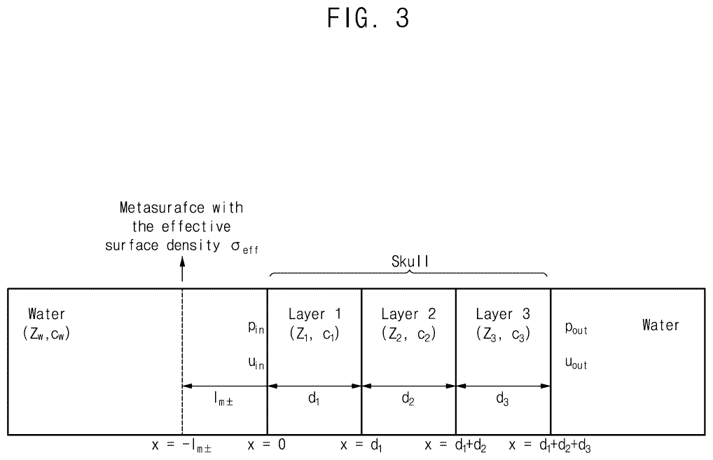

FIG. 3 is a schematic diagram illustrating the impedance matching of a metamaterial with an effective area density placed on water, a medium material, when the ultrasonic wave from the ultrasonic transducer is incident towards water, a medium material, and the skull, which is the multilayer barrier, in the ultrasonic probe of FIG. 1.

Referring to FIG. 3, if there is no metamaterial that satisfies the surface impedance matching conditions, the reflectance (rₗ) at the matching position (x=+l) is as shown in <Equation 3-1> below.

$$\\ \begin{matrix}{r}_{l} = {\vert \frac{{Z}_{l} - {Z}_{w}}{{Z}_{l} + {Z}_{w}} \vert}^{2} & < Equation 3 - 1 >\end{matrix}$$

In addition, the position (l_{m±}) where the real part of the surface impedance becomes equal to the characteristic impedance of water, the medium material, is as shown in <Equation 3-2> below.

$$\\ \begin{matrix}{l}_{m \pm } = \frac{{c}_{w}}{2 ω} arg ( ( 1 \pm i \frac{\sqrt{1 - {\vert {R}_{s} \vert}^{2}}}{\vert {R}_{s} \vert} ) {R}_{s} ) & < Equation 3 - 2 >\end{matrix}$$

Here, |Rₛ|<1.

In addition, the sound pressure reflection coefficient (Rₛ) just in front of the surface of the skull, which is the multilayer barrier, is as shown in <Equation 3-3> below.

$$\\ \begin{matrix}{R}_{s} = \frac{{M}_{11} - {Z}_{w} {M}_{21} + \frac{{M}_{12}}{{Z}_{w}} - {M}_{22}}{{M}_{11} + {Z}_{w} {M}_{21} + \frac{{M}_{12}}{{Z}_{w}} + {M}_{22}} & < Equation 3 - 3 >\end{matrix}$$

In addition, if there is no metamaterial at the matching position (=−l_{m±}), the surface impedance (Zₗ_{_{m±}}) is as shown in <Equation 3-4> below.

$$\\ \begin{matrix}{Z}_{{l}_{m \pm }} = {Z}_{w} ( 1 ∓ i \frac{2 \vert {R}_{s} \vert}{\sqrt{1 - {\vert {R}_{s} \vert}^{2}}} ) & < Equation 3 - 4 >\end{matrix}$$

Here, |Rₛ|<1.

In addition, the impedance (Z_{MS}) of the metamaterial with an effective surface density (σ_{eff}) is as shown in <Equation 3-5> below.

$$\\ \begin{matrix}{Z}_{MS} = i ω {p}_{eff} & < Equation 3 - 5 >\end{matrix}$$

In addition, if there is a metamaterial with an effective area density (σ_{eff}) at the matching position (x=−l_{m±}), the surface impedance (Zₗ_{_{m±}}′) immediately in front of the surface of the metamaterial is as shown in <Equation 3-6> below.

$$\\ \begin{matrix}{Z}_{{l}_{m \pm }}^{′} = {Z}_{{l}_{m \pm }} + {Z}_{MS} = {Z}_{w} ( 1 ∓ i \frac{2 \vert {R}_{s} \vert}{\sqrt{1 - {\vert {R}_{s} \vert}^{2}}} ) + i ω {σ}_{eff} & < Equation 3 - 6 >\end{matrix}$$

Here, |Rₛ|<1.

In addition, in <Equation 3-6>, the reflectance (rₗ_{_{m±}}′) right in front of the surface of the metamaterial is as shown in <Equation 3-7> below.

$$\\ \begin{matrix}{r}_{{l}_{m \pm }}^{′} = {\vert \frac{{Z}_{{l}_{m \pm }}^{′} - {Z}_{w}}{{Z}_{{l}_{m \pm }}^{′} + {Z}_{w}} \vert}^{2} & < Equation 3 - 7 >\end{matrix}$$

In addition, the effective area density (σ_{eff,±}) that must be obtained for impedance matching of the metamaterial to be placed at the matching position (x=−l_{m±}) is as shown in <Equation 3-8> below.

$$\\ \begin{matrix}{σ}_{eff , \pm } = \pm \frac{2 {ρ}_{w} {c}_{w}}{ω} \frac{\vert {R}_{s} \vert}{\sqrt{1 - {\vert {R}_{s} \vert}^{2}}} & < Equation 3 - 8 >\end{matrix}$$

Here, |Rₛ|<1.

In addition, the effective area density (σ_{eff,±}) of the metamaterial may be defined as <Equation 3-9> below.

$$\\ \begin{matrix}{σ}_{eff} ≡ Δ p / {{ξ}^{″}}_{av} & < Equation 3 - 9 >\end{matrix}$$

Here, Δp is the pressure difference between the front and back of the metamaterial, and ξₐᵥ is the surface average acceleration of the metamaterial due to the pressure difference (Δp) between the front and back of the metamaterial.

To elaborate, as can be seen from <Equation 3-1>, to implement impedance matching at the matching position (x=−l) spaced apart from the skull surface (x=O) by the matching distance (l) toward the ultrasonic transducer 1, the surface impedance (Zₗ) of the matching position must be equal to the characteristic impedance (Z_{w}) of the intermediate material (water).

However, in general, except in the case of total reflection (|Rₛ|=1), there is a matching position (x=−l_{m±}) where the real part of the surface impedance (Zₗ) becomes equal to the characteristic impedance of the intermediate material. That is, the matching position (x=−l_{m±}) and the surface impedance (Zₗ_{_{m±}}) at the matching positiong (x=−l_{m±}) can be calculated from <Equation 3-2> and <Equation 3-4>.

At this time, at the corresponding matching position (x=−l_{m±}), an imaginary part generally exists in the surface impedance, as shown in <Equation 3-4>. Therefore, to realize impedance matching at the corresponding matching position (x=−l_{m±}), only the corresponding imaginary part must be removed.

However, as in <Equation 3-5>, when placing the metamaterial with the effective area density (σ_{eff}) at the corresponding matching position (x=−l_{m±}), the surface impedance (Zₗ_{_{m±}}′) immediately in front of the surface of the metamaterial is calculated as <Equation 3-6>, and thus impedance matching can be realized when the metamaterial has the corresponding effective area density (σ_{eff,±}), as shown in <Equation 3-8>.

The metastructure 10 manufactured in this way may be manufactured to have an anti-resonance mode in which the magnitudes of positive and negative vibrations in the natural vibration mode are substantially the same, and the ultrasonic wave may be virtually 100% transparent across the multilayer barrier.

The matching distance (of the metastructure 10 may be actively adjusted to correspond to the surface impedance at the surface 2a of the multilayer barrier 2, and then, the ultrasonic wave may be effectively transmitted through the multilayer barrier 2 depending on environmental conditions. For example, the thickness and physical properties (density, etc.) of the cortical bone and cancellous bone that make up the skull vary depending on the individual and location, and here, in this way, the matching distance e may be actively adjusted according to the conditions of the skull through which ultrasonic wave is transmitted.

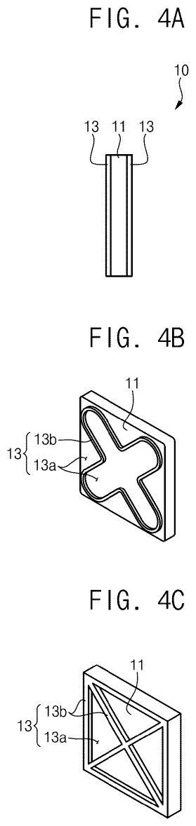

FIG. 4A, FIG. 4B and FIG. 4C are side views and perspective views illustrating the metastructure of FIG. 1.

Referring to FIG. 4A to FIG. 4C, the metastructure 10 includes a first material layer 11 and a second material layer 13.

The first and second material layers 11 and 13 are stacked with each other along the ultrasonic transmission direction.

Here, the first material layer 11 has a first elastic modulus, a first density and a first thickness, and the second material layer 13 has a second elastic modulus, a second density and a second thickness. The first and second elastic moduli may be different from each other, and the first elastic modulus may be larger than the second elastic modulus. The first and second densities may be different from each other.

Alternatively, the first and second elastic moduli may be different and the first and second densities may be same, or the first and second elastic moduli may be same and the first and second densities may be different, or the first and second elastic moduli may be different and the first and second densities may be different.

The first and second thicknesses may be different or same. In addition, a total thickness of the metastructure 10 which is the sum of the first and second thicknesses may be very small compared to the wavelength.

In this way, the metastructure 10 may effectively satisfy the anti-resonance mode from the stacked structure of the first material layer 11 and the second material layer 13.

Further, by controlling the different between the first and second moduli, the different between the first and second densities and the difference between the first and second thicknesses, the anti-resonance mode of the metastructure 10 may be changed. That is, the anti-resonance mode of the metastructure 10 is appropriately adjusted to correspond to the surface impedance on the surface 2a of the multilayer barrier 2, and thus ultrasonic wave may be effectively transmitted through the multilayer barrier 2 depending on various environmental conditions.

As explained above, the metastructure includes the first and second material layers 11 and 13, and as illustrated in FIG. 4A, a pair of second material layers 13 may be disposed on both sides (front and back) of the first material layer 11 with the first material layer 11 interposed therebetween.

Here, as illustrated in FIG. 4B and FIG. 4C, the second material layer 13 may have a pattern unit. Here, the pattern unit may include a first area 13a and a second area 13b.

The first area 13a is an area exposing the first material layer 11 to the outside, and the second area 13b is an area blocking the first material layer 11 without exposing the first material layer 11 to the outside.

For example, the pattern unit may be provided radially with respect to a center point in the transmission direction of the ultrasonic wave. That is, as shown in FIG. 4B and FIG. 4C, the pattern unit is formed in a four-leaf clover shape or an X shape, but the shape of the pattern unit is not limited thereto. However, the first area 13a and the second area 13b included in the pattern unit may have a symmetrical shape with respect to the center of the pattern unit. The pattern unit may be formed in the first material layer 11 in addition to the second material layer 13. The pattern unit formed in the first material layer 11 may be the same or different pattern with the pattern in the second material layer 13.

Accordingly, due to the pattern unit, the anti-resonance mode of the metastructure 10 may be performed more effectively.

Furthermore, by appropriately changing the design of the pattern unit, the anti-resonance mode of the metastructure 10 may be appropriately adjusted to effectively transmit the ultrasonic wave through the multilayer barrier 2 according to various environmental conditions.

As illustrated in FIG. 4A to FIG. 4C, when the pair of the second material layer 13 are formed on both sides (front and back surfaces) of the first material layer 11, the first material layer 11 may include polyimide (P1) and the second material layer 13 may include copper (Cu). Here, the thickness of the first material layer 11 may be in a range between 12 μm and 25 μm, and the second material layer 13 may be in a range between 9 μm and 75 μm.

When the metastructure 10 illustrated in FIG. 4A to FIG. 4C is placed at a position spaced apart from the surface 2a of the multilayer barrier 2 (skull) by the matching distance e toward the ultrasonic transducer 1, in the surface impedance immediately in front of the surface of the metastructure 10 in <Equation 3-6> described above, the size of the real part does not change, and the size of the imaginary part can be changed close to 0 (zero).

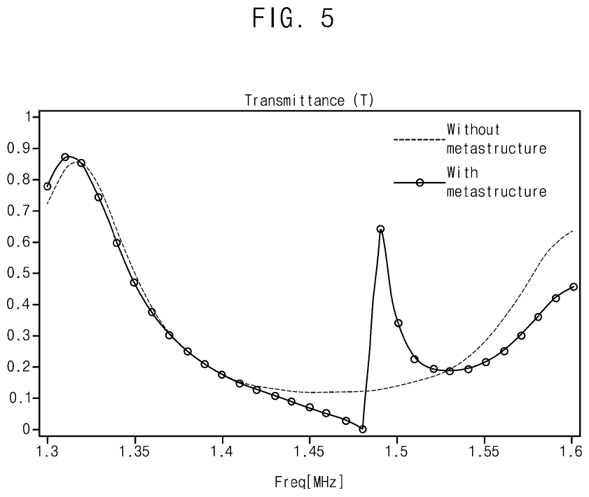

FIG. 5 is a graph showing transmittance by frequency when the metastructure of FIG. 4A is applied and when the metastructure of FIG. 4A is not applied.

As illustrated in FIG. 5, when the metastructure 10 according to the present example embodiment is applied to the frequency range with relatively low transmittance, the transmittance at the frequency range is increased rapidly.

As explained above, the metastructure 10 may be designed to have the predetermined operating frequency, but the operating frequency of the metastructure 10 may be actively changed. For example, by applying thermal or electrical forces are applied to the first material layer 11 or the second material layer 13, at least one of the modulus, the thickness or the pattern unit of each of the first and second material layers 11 and 13, and then the total operating frequency of the metastructure 10 may be changed. Finally, by controlling the operating frequency of the metastructure 10 actively corresponding to the surface impedance at the surface 2a of the multilayer barrier 2, the ultrasonic wave may be effectively transmitted through the multilayer barrier 2, depending on environmental conditions.

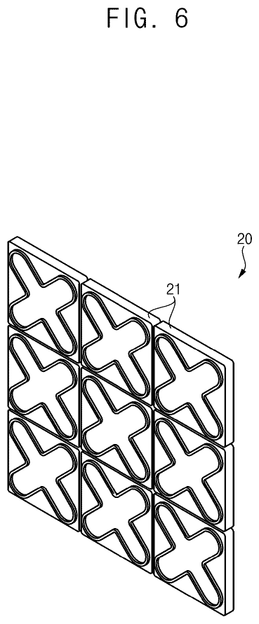

FIG. 6 is a perspective view illustrating a metastructure according to another example embodiment of the present invention.

Referring to FIG. 6, the metastructure 20 according to the present example embodiment includes a plurality of unit metastructures 21. The unit metastructures 21 may be tiled and arranged in a direction crossing the ultrasonic transmission direction to form the metastructure 20. That is, the plurality of unit metastructures 21 is arranged in so-called tiling in a first direction and a second direction perpendicular thereto to form one metastructure group, thereby forming the metastructure 20. Here, the tiling arrangement is a plurality of pieces arranged in the first direction and the second direction, and means that n*m (n and m are natural numbers) unit metastructures 21 are arranged adjacent to each other.

Here, each of the unit metastructures 21 may be the same as the metastructure 10 explained referring to FIG. 4A to FIG. 4C.

Here, neighboring unit metastructures 21 may remain physically coupled to each other, and then the anti-resonance modes of each unit metastructure 21 may be interlocked with each other. Alternatively, the neighboring unit metastructures 21 may remain physically separated, and then the anti-resonance mode of each unit metastructure 21 may be implemented independently.

Additionally, each unit metastructure 21 may be designed to have different operating frequencies, which can achieve a broadband effect of the entire metastructure group 20. Of course, each unit metastructure 21 constituting the entire metastructure group 20 may be designed to have the same operating frequency, and in this case, the operating frequency of each unit metastructure 21 may be actively changed corresponding to the surface impedance on the surface of the multilayer barrier, as described above.

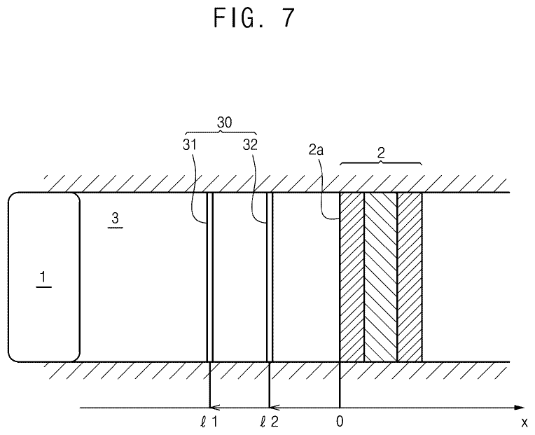

FIG. 7 is a schematic view illustrating a state to which a metastructure according to still another example embodiment of the present invention is applied.

The metastructure 30 according to the present example embodiment includes a first metastructure 31 and a second metastructure 32 along the ultrasonic transmission direction.

Here, each of the first and second metastructures 31 and 32 may be the same as the metastructure 10 as explained referring to FIG. 4A to FIG. 4C.

The first metastructure 31 may be disposed at a position spaced apart from the surface 2a of the multilayer barrier 2 toward the ultrasonic transducer 1 by a first matching distance [symbol] 1, within the intermediate material 3 provided between the ultrasonic transducer 1 and the multilayer barrier 2.

In addition, the second metastructure 32 may be disposed at a position spaced apart from the surface 2a of the multilayer barrier 2 toward the ultrasonic transducer 1 by a second matching distance [symbol] 2, within the intermediate material 3 provided between the ultrasonic transducer 1 and the multilayer barrier 2.

Here, the first matching distance [symbol] 1 may be longer than the second matching distance [symbol] 2. Thus, the first metastructure 31 is located relatively closer to the ultrasonic transducer 1.

In addition, the first metastructure 31 and the second metastructure 32 may each maintain a physically fixed position corresponding to the surface impedance on the surface 2a of the multilayer barrier 2, but as explained above, may be actively changed to correspond to the surface impedance on the surface 2a of the multilayer barrier 2. That is, the first matching distance [symbol] 1 or the second matching distance [symbol] 2 may be actively changed corresponding to the surface impedance at the surface 2a of the multilayer barrier 2.

Accordingly, the metastructure according to the present example embodiment is designed so that the imaginary part of the surface impedance at the matching distance [symbol] is zero (0), and thus the surface impedance at the predetermined matching distance [symbol] becomes the same as the characteristic impedance of the intermediate material 3. Then, the ultrasonic wave may be virtually 100% transparent across the multilayer barrier.

Additionally, in the case of the metastructure, when adjusting the matching distance of the metastructure to correspond to the surface impedance on the surface of the multilayer barrier, or adjusting the physical properties such as the elastic modulus and the pattern unit of the metastructure, the metastructure may effectively transmit the ultrasonic wave while actively coping with the environmental conditions of the multilayer barrier.

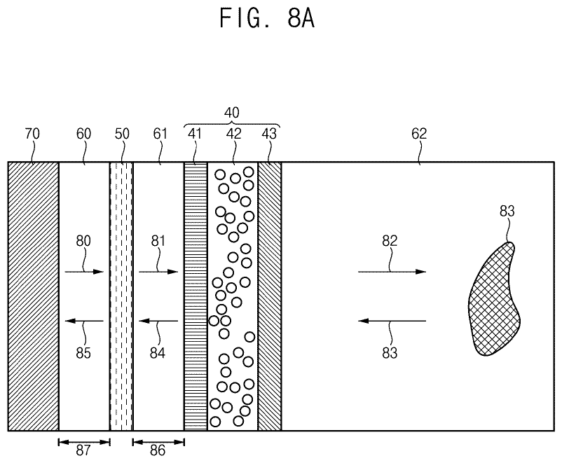

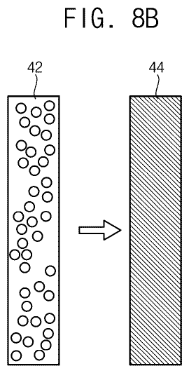

FIG. 8A is a schematic view illustrating a state to which a metastructure according to still another example embodiment of the present invention is applied, and illustrating the metastructure for impedance matching for a barrier with a porous structure.

In the present example embodiment, it is also possible to provide the metastructure that determines the thickness and insertion position (matching distance) of the metastructure by measuring the intensity of the ultrasonic waves reflected or transmitted through the multilayer barrier. This may facilitate impedance matching for the multilayer barrier with non-uniform porous structures where surface impedance calculations are difficult.

FIG. 8A is a schematic diagram illustrating the metastructure 50 for the impedance matching, on the barrier 40 having a dense outer layer or an outer cortical layer 41, an inner porous structure 42, and a dense inner layer or an inner cortical layer 43.

Referring to FIG. 8A, the ultrasonic wave 80 generated from the ultrasonic transducer 70 passes through the metastructure 50, and becomes a transmission-directed ultrasonic wave 81 that passes through the medium 61 between the metastructure 50 and the barrier 40 and propagates. Here, the reflected ultrasonic wave 84 in the receiving direction is generated due to the difference in impedance between the barrier 40 and the medium 61. If the physical properties (impedance) and thickness of the metastructure 50 and the gap 86 between the barrier 40 and the gap 87 between the transducers are appropriately selected, interference between the transmitted and received ultrasonic waves 81 and 84 placed between the metastructure 50 and the barrier 40 may be adjusted, and thus the barrier penetrating the ultrasonic wave 82 may be maximized.

At this time, the theoretically perfect ultrasonic non-reflection condition or perfect ultrasonic transmission condition without considering attenuation due to scattering or absorption is as <Equation 4-1a> and <Equation 4-1b> below, respectively.

$$\\ \begin{matrix}R ≡ \vert \frac{B}{A} \vert = 0 & < Equation 4 - 1 a >\end{matrix}$$

$$\\ \begin{matrix}T ≡ \vert \frac{C}{A} \vert = 1 & < Equation 4 - 1 b >\end{matrix}$$

Here, R refers to the reflectance of the multilayer system consisting of metastructure-intermediate medium-barrier, and is defined as the ratio between the amplitude (pressure field or displacement field) of the ultrasonic wave reflected in the system and the amplitude (pressure field or displacement field) of the ultrasonic wave incident on the system. T refers to the ultrasonic transmittance of the system, and is defined as the size of the ratio between the amplitude C of the ultrasonic wave passing through the system and the amplitude A of the ultrasonic wave incident on the system.

<Equation 4-2a> and <Equation 4-2b> respectively express the case where the amplitude of the incident and reflected ultrasonic waves described above in the incident medium of the multilayer system is the amplitude of the pressure field (usually used in acoustics) and the amplitude of the particle displacement field (V) (usually used in elastography), respectively.

$$\\ \begin{matrix}p = {Ae}^{iwt - ikx} + {Be}^{iwt - ikx} & < Equation 4 - 2 a >\end{matrix}$$

$$\\ \begin{matrix}v = {Ae}^{iwt - ikx} + {Be}^{iwt - ikx} & < Equation 4 - 2 b >\end{matrix}$$

In addition, since the impedance matching phenomenon described above in FIG. 8A has reciprocity, the barrier transmittance of the reflected echo signal 83 of the object 63 may be increased, and finally, the ultrasonic receiving signal 85 including the signal from the object 63 arrives at the ultrasonic transducer 70 and is converted into an electrical signal to be used for image processing.

In order to efficiently select the physical properties, thickness, and insertion position of the metastructure 50 in the present example embodiment, first, let us consider the simplified case where a single-layer metastructure (characteristic impedance z₁, wave number k of thickness d and a single-layer barrier (characteristic impedance z₂, number k₁) of thickness d₁ and a single-layer barrier (characteristic impedance z₂, wave number k₂) of thickness d₂ are placed within the same background medium (characteristic impedance z₀, wave number k₀) at a certain distance l apart.

The transmission coefficient representing the amplitude and phase of ultrasonic waves penetrating the metastructure (i=1) or barrier (i=2) is as shown in <Equation 4-3a>, and the reflection coefficient representing the amplitude and phase of the ultrasonic wave reflected by the metastructure (i=1) or barrier (i=2) is as shown in <Equation 4-3b>.

$$\\ \begin{matrix}{t}_{i} = \frac{2}{2 cos ( {k}_{i} {d}_{i} ) + i ( {z}_{0} / {z}_{i} + {z}_{i} / {z}_{0} ) sin ( {k}_{i} {d}_{i} )} & < Equation 4 - 3 a >\end{matrix}$$

$$\\ \begin{matrix}{r}_{i} = \frac{- i ( {z}_{0} / {z}_{i} - {z}_{i} / {z}_{0} ) sin ( {k}_{i} {d}_{i} )}{2 cos ( {k}_{i} {d}_{i} ) + i ( {z}_{0} / {z}_{i} + {z}_{i} / {z}_{0} ) sin ( {k}_{i} {d}_{i} )} & < Equation 4 - 3 b >\end{matrix}$$

The changes in the ultrasonic pressure field (pᵢ) and particle velocity field (uᵢ) that change when the wave propagates the distance dᵢ in each medium (background medium i=O, metastructure i=1, barrier i=2) are expressed as a transfer matrix Tᵢ as shown in <Equation 4-4> below.

$$\\ \begin{matrix}{( \begin{matrix}{P}_{i} \\ {u}_{i}\end{matrix} )}_{x = {d}_{i}} = {{T}_{i} ( \begin{matrix}{P}_{i} \\ {u}_{i}\end{matrix} )}_{x = 0} & < Equation 4 - 4 a >\end{matrix}$$

$$\\ \begin{matrix}{T}_{i} = ( \begin{matrix}cos ( {k}_{i} {d}_{i} ) & {iz}_{i} sin ( {k}_{i} {d}_{i} ) \\ - \frac{sin ( {k}_{i} {d}_{i} )}{{iz}_{i}} & cos ( {k}_{i} {d}_{i} )\end{matrix} ) & < Equation 4 - 4 b >\end{matrix}$$

$$\\ \begin{matrix}{z}_{i} = {ρ}_{i} {c}_{i} & < Equation 4 - 4 c >\end{matrix}$$

<Equation 4-4c> represents the acoustic characteristic impedance (Zᵢ) expressed as the product of the mass density (ρᵢ) of the medium and the speed of sound (cᵢ).

The above transfer matrix can be expressed as <Equation 4-5> below for the stress field

$$\\ ( {σ}_{xx}^{i} )$$

and particle velocity field (uᵢ) of ultrasonic waves propagating through the multilayer system.

$$\\ \begin{matrix}{( \begin{matrix}{σ}_{xx}^{i} \\ {u}_{i}\end{matrix} )}_{x = {d}_{i}} = {{T}_{i} ( \begin{matrix}{σ}_{xx}^{i} \\ {u}_{i}\end{matrix} )}_{x = 0} & < Equation 4 - 5 a >\end{matrix}$$

$$\\ \begin{matrix}{T}_{i} = ( \begin{matrix}cos ( {k}_{i} {d}_{i} ) & {iz}_{i} sin ( {k}_{i} {d}_{i} ) \\ + \frac{sin ( {k}_{i} {d}_{i} )}{{iz}_{i}} & cos ( {k}_{i} {d}_{i} )\end{matrix} ) & < Equation 4 - 5 b >\end{matrix}$$

$$\\ \begin{matrix}{z}_{i} = {C}_{11}^{i} / {c}_{i} & < Equation 4 - 5 c >\end{matrix}$$

<Equation 4-5c> represents the mechanical characteristic impedance (Zᵢ) expressed as the ratio of the compression/tensile elastic modulus

$$\\ ( {C}_{11}^{i} )$$

of the medium and the speed of sound (cᵢ).

The transfer matrix of acoustics is based on Euler's equation, but the transfer matrix of solid elasticity is based on Hooke's law, so the expressions of <Equation 4-4> and <Equation 4-5> are partially different, but the components of each transfer matrix are all identical in that they are a function of the characteristic impedance (Zᵢ) and phase change (φᵢ=kᵢdᵢ) of the medium, and thus the multilayer barrier penetration metastructure of the present example embodiment may be applied to both acoustic and elastic barrier media.

In the following description, it is explained in terms of acoustics standards to facilitate understanding. From this perspective, the transmission coefficient and reflection coefficient that penetrate the metastructure (i=1) or barrier (i=2) in <Equation 4-3> above were also expressed based on the transfer matrix of <Equation 4-4>.

Using the transfer matrix of the above <Equation 4-4>, ultrasonic pressure field (p) and particle velocity field (u) at the position (x=O) just before and the position (x=D≡d₁+d₀+d₂) immediately after passing the metastructure (i=1), the intermediate medium (i=O) between the metastructure and the barrier, and barrier (i=2), may be expressed as the system transfer matrix (T_{sys}), which is composed of the product of the transfer matrices of the three layers described above, using the following <Equation 4-6>.

$$\\ \begin{matrix}{( \begin{matrix}p \\ u\end{matrix} )}_{x = D} = {{T}_{sys} ( \begin{matrix}p \\ u\end{matrix} )}_{x = 0} & < Equation 4 - 6 a >\end{matrix}$$

$$\\ \begin{matrix}{T}_{sys} = {T}_{2} {T}_{0} {T}_{1} & < Equation 4 - 6 b >\end{matrix}$$

Now, to apply the minimum reflection condition or maximum transmission condition of <Equation 4-1> above, the ultrasonic pressure field and particle velocity field must be expressed as the amplitude variables of the incident, transmitted, and reflected ultrasonic waves of the multilayer system described above. If the amplitude of the ultrasonic wave described above is the amplitude of the pressure field, it may be expressed as <Equation 4-7> below using the well-known linear Euler's equation.

$$\\ \begin{matrix}{( \begin{matrix}p \\ u\end{matrix} )}_{x = 0} = M ( \begin{matrix}A \\ B\end{matrix} ) & < Equation 4 - 7 a >\end{matrix}$$

$$\\ \begin{matrix}{( \begin{matrix}p \\ u\end{matrix} )}_{x = D} = M ( \begin{matrix}{Ce}^{{ik}_{0} D} \\ 0\end{matrix} ) & < Equation 4 - 7 b >\end{matrix}$$

$$\\ \begin{matrix}M = ( \begin{matrix}1 & 1 \\ \frac{1}{{z}_{0}} & \frac{- 1}{{z}_{0}}\end{matrix} ) & < Equation 4 - 7 c >\end{matrix}$$

Then, if the above-mentioned <Equation 4-1>, <Equation 4-6>, and <Equation 4-7> are combined and solved, the conditions for maximizing transmission and minimizing reflection of the multilayer system may be expressed as <Equation 4-8> below. tⱼ and rⱼ are respectively the transmission and reflection coefficients of each medium (metastructure j=1, barrier j=2 mentioned in <Equation 4-3>.

$$\\ \begin{matrix}{e}^{2 {ik}_{0} {d}_{0}} = \frac{{r}_{2}}{{r}_{1}} \{ {( {r}_{1} )}^{2} - {( {t}_{1} )}^{2} \} & < Equation 4 - 8 >\end{matrix}$$

The above <Equation 4-8> may be a design starting condition to approximately and efficiently find the physical properties, thickness, and position of the metastructure that improves the ultrasonic transmittance of the multilayer system.

FIG. 8B is a schematic view showing the approximation of the porous structure within a multilayer barrier with a uniform material in FIG. 8A. This may be another way to efficiently find the physical properties, thickness, and location of the metastructure of the present invention that improves the ultrasonic transmittance of the multilayer system.

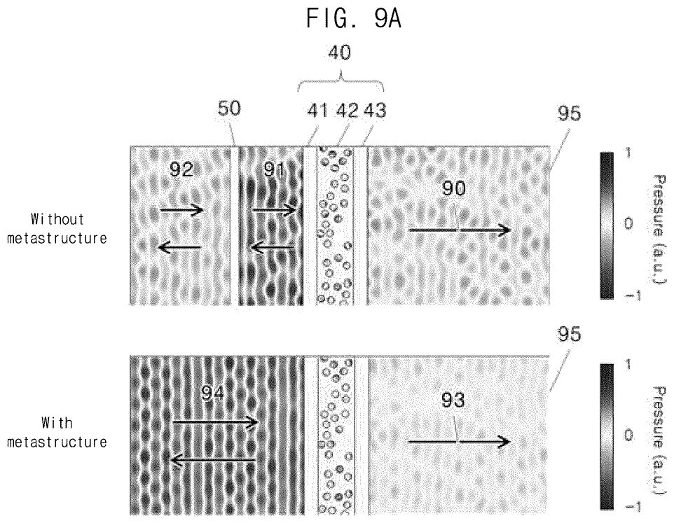

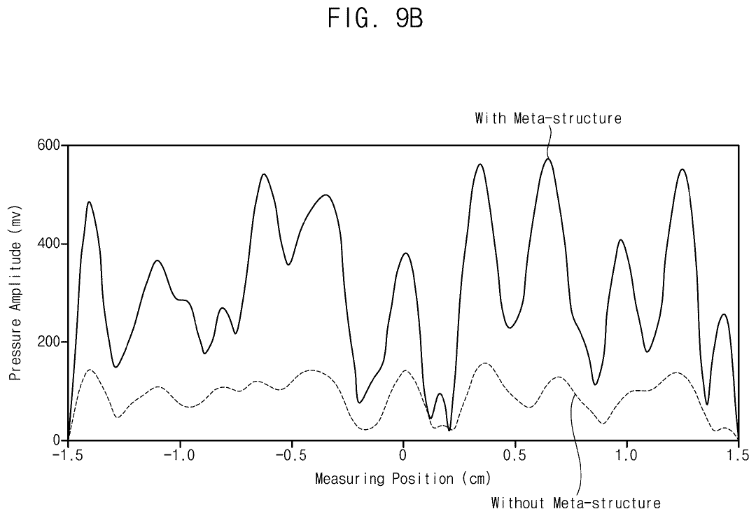

FIG. 9A is an image showing a result of simulation verification that ultrasonic transmission is improved when the metastructure for impedance matching is applied to the barrier having the porous structure of FIG. 8A.

In FIG. 9A, the barrier 40 has the same configuration as that in FIG. 8A, and the metastructure 50 located on the left side of the barrier 40 is positioned at an appropriate thickness or an appropriate distance from the barrier 40.

The upper portion of FIG. 9A shows the case where the metastructure 50 exists. Constructive interference of transmitted and reflected waves in the area 91 between the metastructure 50 and the multilayer barrier 40 is strengthened, and although not shown in the figure, the multiple internal reflection phenomenon intensifies even within the multilayer barrier 40. Here, similar to what is expressed in <Equation 4-6>, the system transfer matrix (T_{sys}) of the multilayer barrier, the metastructure, and the intermediate material minimizes the amplitude or reflection coefficient of the ultrasonic pressure field reflected to the left from the metastructure 50, and maximizes the amplitude or transmission coefficient of the pressure field of the ultrasonic wave 90 penetrating from the barrier 40 to the right. This may be summarized and expressed as <Equation 4-8> below.

$$\\ \begin{matrix}( \begin{matrix}{Ce}^{{ik}_{0} D} \\ 0\end{matrix} ) = {{M}^{- 1} ( \begin{matrix}p \\ u\end{matrix} )}_{x = D} = {M}^{- 1} {{T}_{sys} ( \begin{matrix}p \\ u\end{matrix} )}_{x = 0} = {M}^{- 1} {T}_{sys} M ( \begin{matrix}A \\ B\end{matrix} ) & < Equation 4 - 8 a >\end{matrix}$$

$$\\ \begin{matrix}S ≡ {M}^{- 1} {T}_{sys} M & < Equation 4 - 8 b >\end{matrix}$$

$$\\ \begin{matrix}( \begin{matrix}t \\ 0\end{matrix} ) = S ( \begin{matrix}1 \\ r\end{matrix} ) & < Equation 4 - 8 c >\end{matrix}$$

The above equation is the result of combining the results of <Equation 4-6> and <Equation 4-7>, and may be applied to a system containing an arbitrary multilayer barrier and a metastructure.

In the present example embodiment, by adjusting the thickness, spacing, and physical properties of the metastructure, in the scattering matrix S≡M⁻¹T_{sys}M (transmission matrix for the amplitude of incident, transmission, and reflected waves) of the multilayer system, the transmission coefficient t of the system in <Equation 4-8c> may be adjusted to increase and the reflection coefficient r to be lowered.

The thickness of the metastructure 50, verified by simulation in FIG. 9A, was about 4 mm, and the distance from the multilayer barrier 40 was about 10 mm. In addition, in FIG. 9A, the ultrasonic generator on the left is omitted, and it can be seen that the pressure increases by about three times more in the case where the metastructure 50 is present than in the case without the metastructure, when calculating the average pressure at the right boundary line 95.

FIG. 9B is a graph showing a change in pressure distribution along the right border of FIG. 9A due to the porous structure of the metastructure of FIG. 8A.

As illustrated in FIG. 9B, due to the porous tissue 42, the pressure distribution along the right border 95 changes and is non-uniform, it can be seen that the pressure field when the metastructure 50 is present is larger than when the metastructure is not present. The physical properties of the multilayer barrier 40 used in the simulation were 2 to 4 times greater than the characteristic impedance of the background medium.

On the other hand, if the porous structure within the multilayer barrier is known, as illustrated in FIG. 8B, metastructure design may be facilitated with the transfer matrix pressure field or transmission coefficient, reflection coefficient, or simplified simulation model described above using well-known homogenization methods.

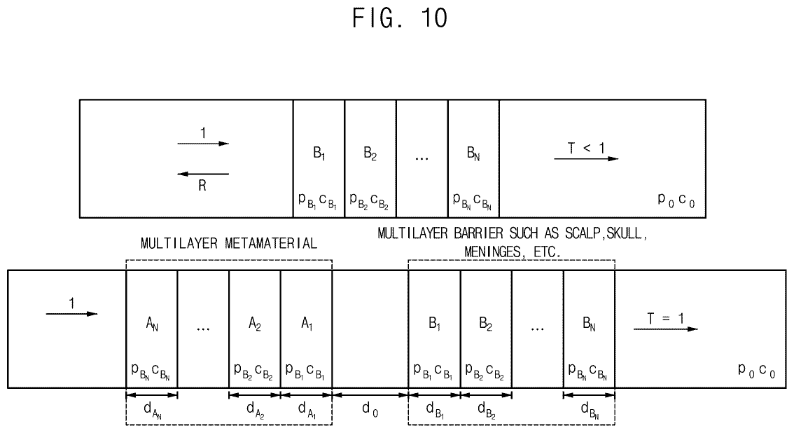

FIG. 10 is a schematic view illustrating a metastructure for impedance matching for multilayer for the multi barrier according to still another example embodiment of the present invention.

FIG. 10 is a schematic view illustrating the multilayer metastructure (A1, A2, . . . , AN layers), as the metastructure for multilayer impedance matching for the multilayer barrier, that facilitates finding impedance matching conditions for N layers of barriers (B1, B2, . . . , BN layers) such as the skull, scalp, meninges, etc.

For perfect ultrasonic penetration through the multilayer barrier, the properties of the multilayer metastructure may be arranged in mirror symmetry with the properties of the multilayer barrier, and this satisfies the impedance matching condition of <Equation 5-1a> and the phase matching condition of <Equation 5-1b>.

$$\\ \begin{matrix}{z}_{Bi} = {z}_{Ai} ( i = 1 , 2 , … , N ) & < Equation 5 - 1 a >\end{matrix}$$

$$\\ \begin{matrix}{k}_{Bi} {d}_{Bi} = {k}_{Ai} {d}_{Ai} ( i = 1 , 2 , … , N ) & < Equation 5 - 1 b >\end{matrix}$$

By using the above-described transfer matrix, the ultrasonic pressure and displacement field when penetrating the multilayer metastructure and multilayer barrier may be calculated, and here the transfer matrix of the multilayer metastructure and the transfer matrix of the multilayer barrier are as shown in <Equation 5-2> below.

$$\\ \begin{matrix}{T}_{A} = [ \begin{matrix}{T}_{A 11} & {T}_{A 12} \\ {T}_{A 21} & {T}_{A 22}\end{matrix} ] = {T}_{1}^{{{d}_{A}}_{1}} {T}_{2}^{{{d}_{A}}_{2}} … {T}_{N - 1}^{{{d}_{A}}_{N - 1}} {T}_{N}^{{{d}_{A}}_{N}} & < Equation 5 - 2 a >\end{matrix}$$

$$\\ \begin{matrix}{T}_{B} = [ \begin{matrix}{T}_{B 11} & {T}_{B 12} \\ {T}_{B 21} & {T}_{B 22}\end{matrix} ] = {T}_{1}^{{{d}_{B}}_{1}} {T}_{2}^{{{d}_{B}}_{2}} … {T}_{N - 1}^{{{d}_{B}}_{N - 1}} {T}_{N}^{{{d}_{B}}_{N}} & < Equation 5 - 2 b >\end{matrix}$$

If we apply the mirror symmetric multilayer metastructure matching condition of <Equation 5-1> to the above transfer matrix, the two transfer matrices have a transpose relationship (T_{A}=T_{B}^{T}), and the relationship between the scattering matrix S and transmission and reflection coefficients of the entire system, including the intermediate cavity medium (transfer matrix T_{C}) between the multilayer metastructure and the multilayer barrier, is as shown in <Equation 5-3> below.

$$\\ \begin{matrix}( \begin{matrix}t \\ 0\end{matrix} ) = S ( \begin{matrix}1 \\ r\end{matrix} ) & < Equation 5 - 3 a >\end{matrix}$$

$$\\ \begin{matrix}S ≡ {M}^{- 1} {T}_{B} {T}_{C} {T}_{A} M = {M}^{- 1} {T}_{B} {{T}_{C} ( {T}_{B} )}^{T} M & < Equation 5 - 3 b >\end{matrix}$$

Here, if <Equation 4-1>, which is the perfect ultrasonic transmission condition described above, is applied to the above equation, it is possible to obtain conditions for how far apart the multilayer metastructure should be inserted from the multilayer barrier, and this is the same as <Equation 5-4> below.

$$\\ \begin{matrix}{d}_{0} = {k}_{0}^{- 1} {tan}^{- 1} ( \frac{i 2 {z}_{0} {T}_{B 11} {T}_{B 12} - i 2 {z}_{0}^{- 1} {T}_{B 21} {T}_{B 22}}{{T}_{B 11}^{2} - {T}_{B 22}^{2} + {z}_{0}^{2} {T}_{B 12}^{2} - {z}_{0}^{- 2} {T}_{B 21}^{2}} ) & < Equation 5 - 4 >\end{matrix}$$

When it is difficult to know the actual number of layers of the barrier, or when the number of layers of the barrier is too large to determine or fabricate the number of layers of the metastructure, by inserting a small number of the metastructures and adjusting their relative positions, the transfer matrix (or scattering matrix) of the entire system is adjusted and thus reflectance may be minimized and transmittance maximized as explained in the previous example embodiment.

For example, the design conditions for increasing ultrasonic multilayer barrier penetration by inserting two metastructures are expressed by the transmission matrix described above, and may be expressed as <Equation 5-5> below.

$$\\ \begin{matrix}( \begin{matrix}t \\ 0\end{matrix} ) = {M}^{- 1} {T}_{M 1} {T}_{C 1} {T}_{M 2} {T}_{C 2} {T}_{B} M ( \begin{matrix}1 \\ r\end{matrix} ) & < Equation 5 - 5 >\end{matrix}$$

The metastructure insertion form of <Equation 5-5> is similar to that shown in FIG. 7. Adjusting the physical properties and thickness of the first and second metastructures 31 and 32 of FIG. 7 is equivalent to adjusting the transfer matrices T_{M1} and T_{M2}, respectively. Adjusting the spacing between the first and second metastructures 31 and 32 and the spacing between the second metastructure 32 and the multilayer barrier 2 is the same as adjusting the transfer matrices T_{C1} and T_{C2}, respectively.

As a result of verification through simulation as shown in FIG. 9A and FIG. 9B, the physical properties (characteristic impedance) of the metastructure that provides impedance matching for the above-described multilayer barrier must have the impedance that is at least twice as large as that of the background medium, in order to see the effect of improving ultrasonic penetration. It is advantageous for impedance matching if the physical properties of the metastructure are similar to or greater than those of the multilayer barrier. This is because it is easy to control the transfer matrix of the entire multilayer system by deepening the multiple internal reflections between the metastructure and the multilayer barrier.

In addition, the metastructure according to the present example embodiment may have vibration modes such as monopole, dipole, quadrupole, etc., and may have negative mass or negative stiffness properties. The role of the metastructure with the corresponding vibration mode or negative physical properties is the same as minimizing the ultrasonic reflectance by controlling the transmission matrix of the entire multilayer system as described above.

In the metastructure 10 according to the previous example embodiments of FIG. 1 and FIG. 2, the impedance matching for the multilayer barrier 2 whose physical properties change discontinuously along the direction in which the ultrasonic wave is transmitted was explained.

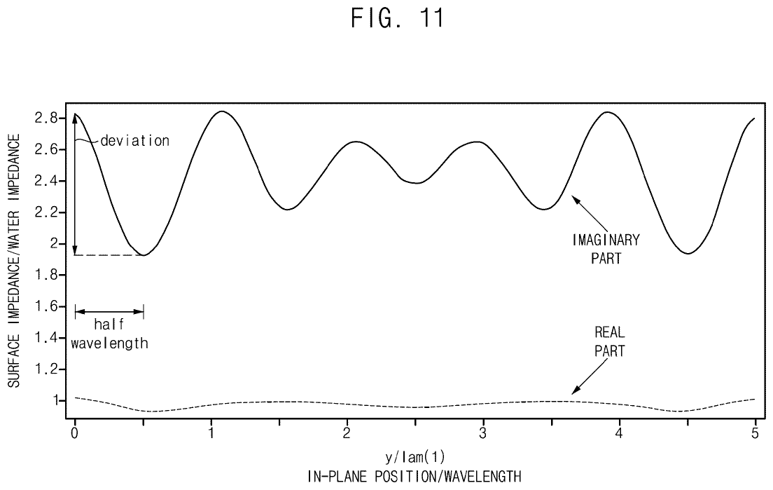

Alternatively, FIG. 11 is a graph showing wavelength characteristics of surface impedance at a location spaced apart by a matching distance from the surface of a non-uniform barrier whose physical properties continuously change along the direction intersecting the direction of ultrasonic transmission.

Here, as illustrated in FIG. 11, in the case of a non-uniform barrier 2 whose physical properties such as density and porosity in the second direction D2 crossing the first direction D1 in which the ultrasonic wave is transmitted continuously change like the skull (cancellous bone), uneven deviation of the surface impedance output value at the predetermined matching distance (of the metastructure 10′ occurs, since the physical properties such as density and porosity of the barrier 2 in the second direction D2 change.

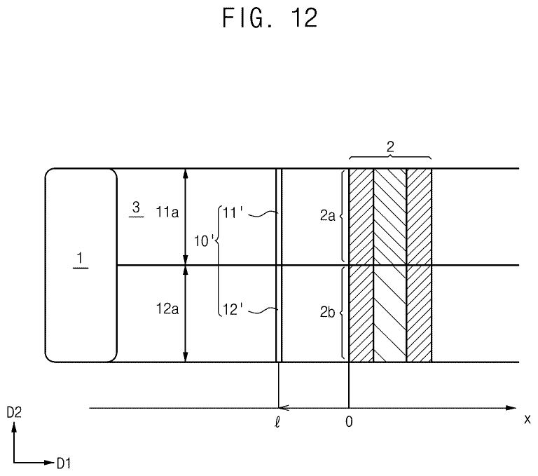

FIG. 12 is a schematic view illustrating a metastructure according to still another example embodiment of the present invention.

Referring to FIG. 12, in the metastructure 10′ according to the present example embodiment, for the non-uniform barrier 2 having different physical properties not only in the first direction D1 through which the ultrasonic wave is transmitted but also in the second direction D2 crossing the first direction D1, the imaginary part of the surface impedance at the matching distance € is made to be zero (0) so that the surface impedance at the predetermined matching distance (becomes the same as the characteristic impedance of the intermediate material 3. Thus, it is possible to reduce the non-uniform deviation of the surface impedance output value at the predetermined matching distance [symbol] of the metastructure 10′ with respect to the substantially non-uniform barrier 2, and to increase transmittance.

The metastructure 10′ includes a first metastructure 11′ and a second metastructure 12′.

The first and second metastructures 11′ and 12′ are disposed inside of the intermediate material 3 between the ultrasonic transducer 1 and the multilayer barrier 2, and are disposed adjacent to each other along the second direction D2.

The first metastructure 11′ is arranged to be spaced apart from the first surface area 2a of the multilayer barrier 2 by a first matching distance (toward the ultrasonic transducer 1, and may cause the ultrasonic wave radiated from the ultrasonic transducer 1 to enter the first surface area 2a of the barrier 2. The first metastructure 11′ may have a first effective radiation area 11a corresponding to the first surface area 2a.

In the first metastructure 11′, the real part of the first surface impedance at the first matching distance [symbol] with respect to the first surface area 2a of the barrier 2 may remain unchanged and the imaginary part may change close to zero (0). Then, the first surface impedance at the first matching distance [symbol] may be substantially equal to the characteristic impedance of the intermediate material 3.

The second metastructure 12′ is arranged to be spaced apart from the second surface area 2b adjacent to the first surface area 2a of the barrier 2 by a second matching distance [symbol] toward the ultrasonic transducer 1, and may cause the ultrasonic wave radiated from the ultrasonic transducer 1 to enter the second surface area 2b of the barrier 2. The second metastructure 12′ may have a second effective radiation area 12a corresponding to the first surface area 2a. Here, the second matching distance [symbol] is substantially same as the first matching distance [symbol] .

In the second metastructure 12′, the real part of the second surface impedance at the second matching distance (with respect to the second surface area 2a of the barrier 2 may remain unchanged and the imaginary part may change close to zero (0). Then, the second surface impedance at the second matching distance [symbol] may be substantially equal to the characteristic impedance of the intermediate material 3.

Accordingly, through the first surface impedance by the first meta structure 11′ and the second surface impedance by the second meta structure 12′, by taking into account changes in the different physical properties of the first surface area 2a and the second surface area 2b of the multilayer barrier 2, it is possible to achieve high transmittance for the non-uniform barrier 2.

Each of the first effective radiation area 11a of the first metastructure 11′ and the second effective radiation area 12a of the second metastructure 12′ may be designed to have a width smaller than the wavelength λ.

Basically, the multilayer barrier 2, such as the skull (cancellous bone), continuously changes its physical properties (density, porosity, etc.) in the second direction (D2) crossing the first direction (D1) through which the ultrasonic wave penetrate. Considering this, the width of the first effective radiation area 11a of the first metastructure 11′ and the second effective radiation area 12a of the second metastructure 12′ are set to be smaller than the wavelength λ, and thus the variation of surface impedance within each effective radiation area may be reduced.

Each of the first effective radiation area 11a of the first metastructure 11′ and the second effective radiation area 12a of the second metastructure 12′ may be set to be smaller than ½ wavelength λ.

According to the continuously changing physical properties (density, porosity, etc.) of the non-uniform barrier (2), at least one of the matching distance, physical properties, and effective radiation area of the first metastructure layer 11′ or the second metastructure layer 12′ may be changed. Then, the ultrasonic wave may be effectively transmitted through the non-uniform barrier 2 according to various environmental conditions.

Accordingly, in the metastructure according to the present example embodiment, the imaginary part of the surface impedance at the matching distance € is designed to be zero (0), so that the surface impedance at the predetermined matching distance (becomes the same as the characteristic impedance of the intermediate material 3. Then, the ultrasonic wave may be transmitted virtually 100% across the non-uniform barrier.

FIG. 13 is a schematic view illustrating a metastructure according to still another example embodiment of the present invention.

In the metastructure according to the present example embodiment, the thickness and insertion location of the metastructure may be determined by measuring the intensity of the ultrasonic wave reflected or transmitted through the barrier having non-uniform thickness and physical properties. This may facilitate impedance matching for the multilayer barrier with a non-uniform porous structure where surface impedance calculations are difficult.

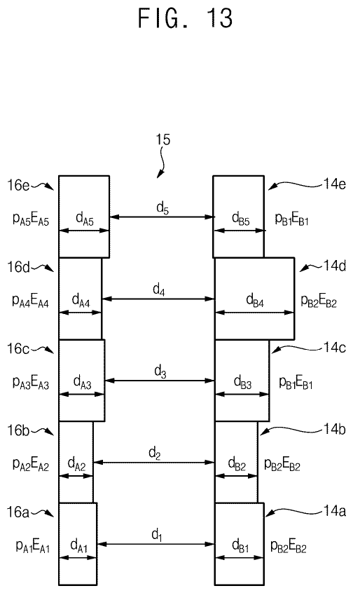

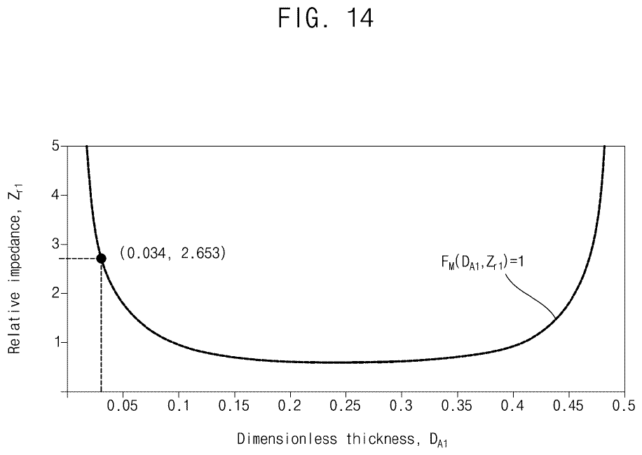

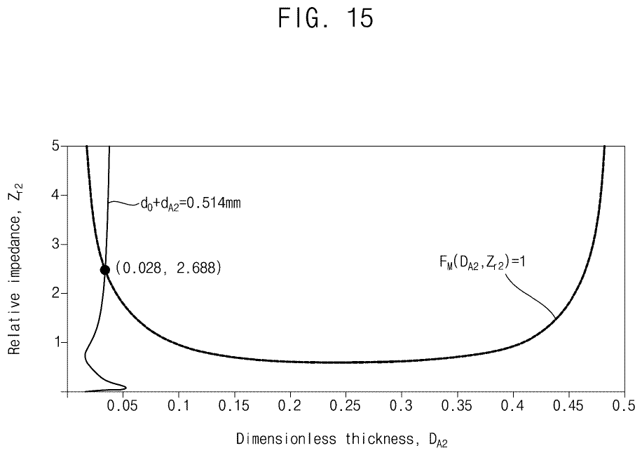

FIG. 14 is a graph showing impedance matching conditions for designing the A1 layer of FIG. 13. FIG. 15 is a graph showing impedance matching conditions for designing the A2 layer of FIG. 13.

Referring to FIG. 13 to FIG. 15, regarding the metastructure according to the present example embodiment, the material of barriers B1 (14a), B2 (14b), and B4 (14d) was set to aluminum, and the material of barriers B3 (14c) and B5 (14e) was set to stainless steel. Each barrier thickness was set to B1=1.2 mm, B2=1.0 mm, B3=1.3 mm, B4=1.7 mm, and B5=1.2 mm. It is assumed that the background mediator 15 is water. Through impedance matching for each barrier zone, the effective density, effective Young's modulus, and thickness, which are effective physical properties, are obtained as follows.

Here, the physical properties corresponding to each barrier, the background intermediate material 15, and the metastructure (impedance matching layer) A1 (16a) are as follows.

Background intermediate material 15: ρₒ=1,000 kg/m3, cₒ=1,500 m/s
Barrier B1 (14a), B2 (14b), and B4 (14d): β_{B}=8,000 kg/m3, E=193 GPa
Barrier B3 (14c), B5 (14e): ρ_{B}=2,700 kg/m3, E=72 GPa

Metastructure (impedance matching layer): β_{B}=8,000 kg/m3, E=193 GPa

$$\\ \begin{matrix}{F}_{M} ( {D}_{A 1} , {Z}_{r 1} ) = \vert \frac{{\overline{Z}}_{OB} sin ( {φ}_{B} )}{{\overline{Z}}_{OA} sin ( {φ}_{A 1} )} \vert \frac{{\overline{Z}}_{{OA}^{2}} sin ( {φ}_{A 1}^{2} ) + 4}{\sqrt{4 {cos}^{2} ( {ϕ}_{A 1} ) + {\tilde{Z}}_{{OB}^{2}} sin ( {φ}_{B}^{2} )} \sqrt{4 {cos}^{2} ( {ϕ}_{A 1} ) + {\tilde{Z}}_{OA {1}^{2}} sin ( {φ}_{A 1}^{2} )}} & < Equation 6 - 1 >\end{matrix}$$

$$\\ \begin{matrix}{Z}_{r 1} = \frac{{Z}_{A 1}}{{Z}_{B 1}} = \frac{\sqrt{{ρ}_{A 1} {c}_{A 1}}}{\sqrt{{ρ}_{B 1} {c}_{B 1}}} & < Equation 6 - 2 >\end{matrix}$$

$$\\ \begin{matrix}{d}_{A 1} = {D}_{A 1} {λ}_{A 1} & < Equation 6 - 3 >\end{matrix}$$

$${d}_{1} ( {D}_{A 1} , {Z}_{A 1} ) = \frac{{c}_{0}}{2 ω} {tan}^{- 1} ( \frac{2 {\tilde{Z}}_{OB} cos {ϕ}_{A 1} sin {ϕ}_{B 1} + 2 {\tilde{Z}}_{0 A 1} sin {ϕ}_{A 1} cos {ϕ}_{B 1}}{{\tilde{Z}}_{0 A 1} {\tilde{Z}}_{OB} sin {ϕ}_{A 1} sin {ϕ}_{B 1} - 4 cos {ϕ}_{A 1} cos {ϕ}_{B 1}} ) \qquad < Equation 6 - 4 >$$

Using the impedance matching condition of <Equation 6-1>, a series of relative impedance (Zᵣ=Z_{A}/Z_{B}) and dimensionless thickness (D_{A}=d_{A}λ_{A}) points with a transmittance of 100% were calculated and shown as a curve (dotted line) in FIG. 14.

The relative impedance and thickness of metastructure A1 (16a) calculated using <Equation 6-2> and <Equation 6-3> are Zᵣ₁=2.653 mm and d_{A1}=0.383 mm, respectively. The distance between the metastructure A1 (16a) and the barrier B1 (14a) obtained through <Equation 6-4> phase matching condition is d₁=0.131 mm.

In order to design the metastructure A2 (16b), not only must the impedance matching condition be satisfied, but also the condition d_{A2}+d₂=d_{A1}+d₁ must be satisfied, as shown in FIG. 15.

If we overlay this on the Impedance matching condition graph, as illustrated in FIG. 5, Zᵣ₁=2.688, D_{A2}=0.028. In the same way as A1 (16a), the effective physical properties and dimensions of metastructure A2 (16b) calculated through <Equation 6-1> to <Equation 6-4> are ρ_{A2}=7,603 kg/m3, E_{A2}=203.3 GPa, d₂=0.158 mm, d_{A2}=0.356 mm.

Similar to the method of designing metastructure A2 (16b), the calculated effective properties and dimensions of each metastructure A3 (16c), A4 (16d), and A5 (16e) are as follows.

$$\\ {ρ}_{A 3} = 20 , 220 kg / m 3 , {E}_{A 3} = 540.7 GPa , {d}_{3} = 0.041 mm , {d}_{A 3} = 0.473 mm , \\ {ρ}_{A 4} = 9 , 597 kg / m 3 , {E}_{A 4} = 256.7 GPa , {d}_{4} = 0.091 mm , {d}_{A 4} = 0.423 mm , \\ {ρ}_{A 5} = 19 , 135 kg / m 3 , {E}_{A 5} = 511.7 GPa , {d}_{5} = 0.045 mm , {d}_{A 5} = 0.469 mm$$

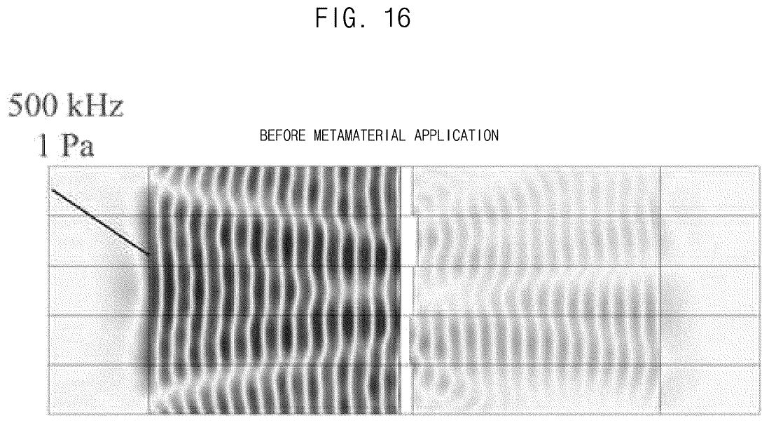

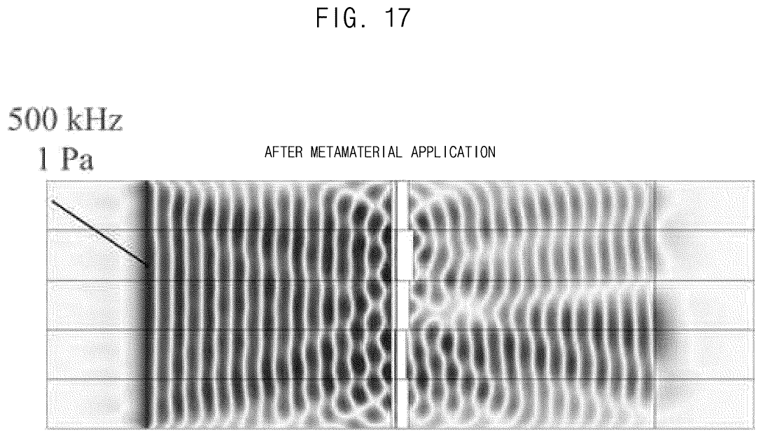

FIG. 16 is a simulated result showing transmittance before applying the metastructure. FIG. 17 is a simulated result showing transmittance after applying the metastructure according to the example embodiments of the present invention.

To check the ultrasonic transmission performance of the metastructure, COMSOL Multiphysics, a commercial simulation program, was used. The effective properties and dimensions of each metastructure layer calculated previously were applied to the model, the ultrasonic penetration pressure before applying the metastructure is shown in FIG. 16, and the ultrasonic penetration pressure after applying the metastructure is shown in FIG. 17.

As in FIG. 16, before applying the metastructure, a pressure of 500 kHz and 1 Pa was applied to the excitation part 16, and the incident wave 17 penetrates the non-uniform barrier 19 to form a transmission wave 18.

As in FIG. 17, after applying the metastructure, a pressure of 500 kHz and 1 Pa was similarly applied to the excitation part 20, the metastructure layer 22 was installed in front of the heterogeneous barrier 23, and the ultrasonic incident wave 21 passed through the metastructure 24 and the heterogeneous barrier 23 to form a transmission wave.

Referring to FIG. 16 and FIG. 17, the waveforms before and after the metastructure were similar, but an average increase in pressure amplitude of about 2.5 times was confirmed, and it may be confirmed that uniform propagation is possible after penetrating an uneven barrier through phase matching according to geometry.

The metastructure explained above is applied to an ultrasonic probe, and hereinafter the ultrasonic probe having the metastructure is explained.

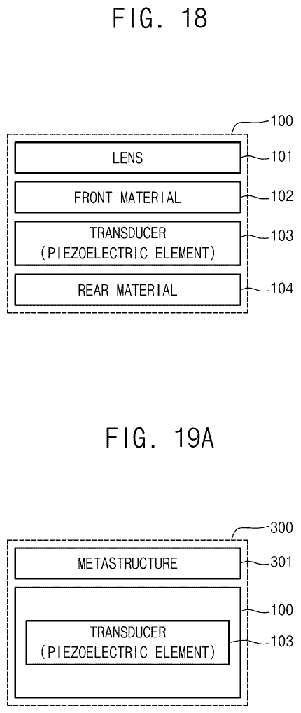

FIG. 18 is a block diagram illustrating the conventional ultrasonic probe.

Referring to FIG. 18, the conventional ultrasonic probe includes an ultrasonic transducer 103 (piezoelectric element) generating the ultrasonic wave, a front material 102 matching the impedance difference between the ultrasonic transducer 103 and soft tissue, a lens 101, and a rear material 104 absorbing the received ultrasonic wave or protecting the ultrasonic transducer 103 from overheating. The lens 101 helps obtain a two-dimensional ultrasonic cross-sectional image by focusing the ultrasonic beam in a direction (elevation) perpendicular to the array direction of the ultrasonic transducer 103.

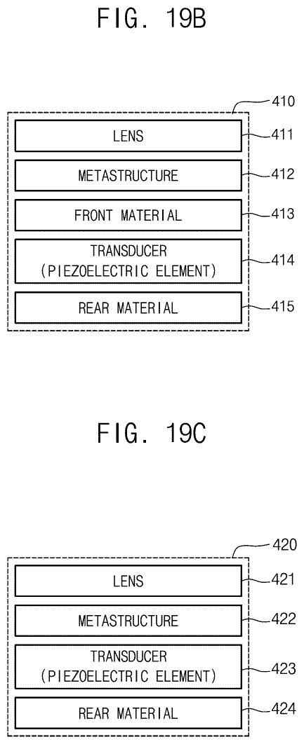

FIG. 19A is a block diagram illustrating an ultrasonic probe according to still another example embodiment of the present invention.

Referring to FIG. 19A, in the ultrasonic probe 300 according to the present example embodiment, the metastructure 301 according to the previous example embodiments is manufactured as a separate module from the conventional ultrasonic probe 100 and is positioned in front (above in the drawing) of the conventional ultrasonic probe 100. This provides the multilayer barrier and additional impedance matching.

FIG. 19B is a block diagram illustrating an ultrasonic probe according to still another example embodiment of the present invention.

Referring to FIG. 19B, in the ultrasonic probe 410 according to the present example embodiment, the metastructure 412 according to the previous example embodiments is included in the ultrasonic probe 410, and functions as a component located between the outermost (front) lens 411 and the front material 413. Here, the front material 413 must implement impedance matching between the piezoelectric element 414 and the metastructure 412, which has a large acoustic impedance, so its physical properties may be different from the configuration of the front material of the conventional ultrasonic probe. Since the front material of the conventional ultrasonic probe is mainly manufactured by mixing resin with metal or ceramic powder, it is possible to match the difference in acoustic impedance between the ultrasonic transducer (piezoelectric element) and the metastructure by varying the composition ratio.

FIG. 19C is a block diagram illustrating an ultrasonic probe according to still another example embodiment of the present invention.

Referring to FIG. 19C, in the ultrasonic probe 420 according to the present example embodiment, the metastructure 422 according to the previous example embodiments replaces the front material within the ultrasonic probe 420 to perform impedance matching between the ultrasonic transducer 423 (piezoelectric element) and the multilayer barrier.

Here, although not shown in the figure, if the metastructure 422 is bent in a direction perpendicular to the array direction of the ultrasonic transducer 423 or has different physical properties or shapes, the metastructure 422 may replace the role of the lens 421 in the ultrasonic probe 420.

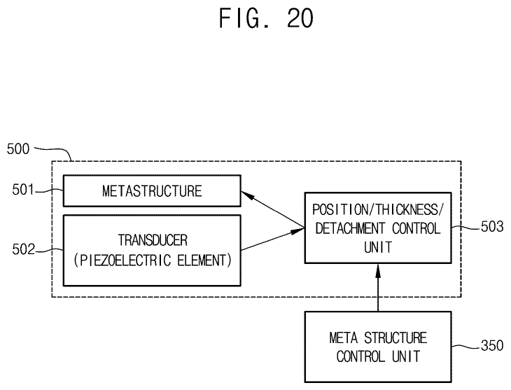

FIG. 20 is a block diagram for explaining a control of the metastructure, in an ultrasonic probe according to still another example embodiment of the present invention.

Referring to FIG. 20, as explained above, in order for the metastructure 501 according to the present example embodiment to effectively perform impedance matching for any multilayer barrier, the insertion position (or relative distance to the multilayer barrier or the probe) of the metastructure 501 and the thickness or physical properties of the metastructure 501 may be changed through the position/thickness/attachment/detachment control unit 503 included in the ultrasonic probe 500.

That is, the position/thickness/attachment/detachment control unit 503 may change the thickness or physical properties of the metastructure 501 by generating external force mechanically, thermally, or electromagnetically. In addition, the position/thickness/detachment control unit 503 may include a motor mechanism or a joint mechanism to enable attachment and detachment of the metastructure 501 to replace it with another metastructure.

In addition, the position/thickness/attachment/detachment control unit 503 may control the insertion position and distance of the metastructure 501. In addition, the position/thickness/attachment/detachment control unit 503 may actually control the metastructure 501 by receiving commands from the external metastructure control unit 350 through a probe cable, etc. Details regarding this metastructure control will be further explained when describing the ultrasonic image diagnosis apparatus described later.

The metastructure explained in the previous example embodiments may be applied to an ultrasonic image diagnosis apparatus, and hereinafter the ultrasonic image diagnosis apparatus having the metastructure is explained.

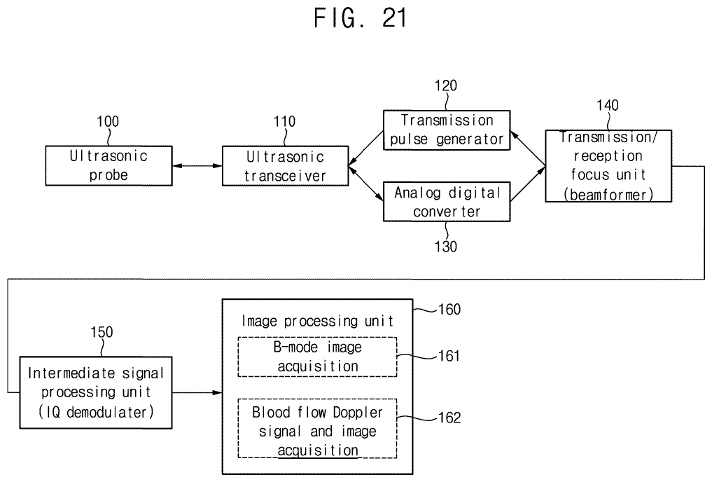

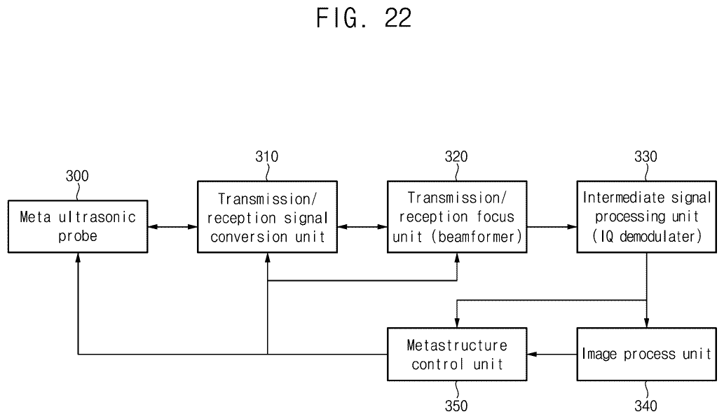

FIG. 21 is a block diagram illustrating the conventional ultrasonic image diagnosis apparatus.

Referring to FIG. 21, the conventional ultrasonic image diagnosis apparatus includes an ultrasonic transceiver 110 transmitting an ultrasonic analog signal converted to electricity with the ultrasonic probe 100 or amplifying the received signal, a transmission pulse generator 120, an analog digital converter 130 converting the received analog signal, a transmission/reception focus unit 140 that adjusts the time delay for each channel to form a wavefront of the signal transmitted to the digital converter array or to specify the signal received for each channel by image location, an intermediate signal processing unit 150 that lowers the sampling rate and converts real-valued received signals into complex-valued signals to facilitate image processing, and an image processing unit 160 that converts channel signals into B-mode images (161) or extracts blood flow signals and converts them into sound or images 162.

FIG. 22 is a block diagram illustrating an ultrasonic image diagnosis apparatus according to still another example embodiment of the present invention.

Referring to FIG. 22, the ultrasonic image diagnosis apparatus according to the present example embodiment include a meta ultrasonic probe 300 and a metal structure control unit 350, unlike the conventional ultrasonic image diagnosis apparatus.

The metastructure control unit 350 may determine the degree of impedance matching through the ultrasonic reflectance of the current multilayer barrier and metastructure system through the ultrasonic receiving signal. In addition, the meta structure control unit 350 may calculate the effect of the meta-structure on the image based on the received ultrasonic channel signal and the current image result synthesized through the well-known Delay-And-Sum (DAS) technique. Here, the effect of the meta-structure includes whether image brightness is improved, signal-to-noise ratio is improved, axial resolution is reduced due to multiple internal reflections within the metastructure and multilayer barrier, and signal coherency is reduced due to ultrasonic refraction of the metastructure.

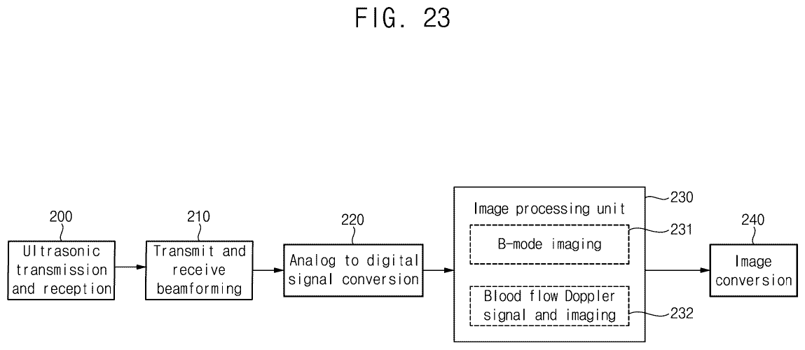

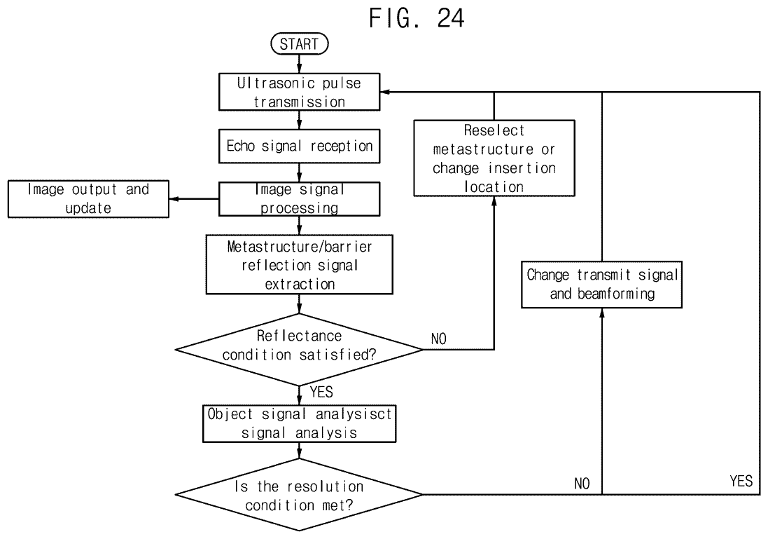

FIG. 23 is a block diagram for explaining an operation method of the ultrasonic image diagnosis apparatus of FIG. 21.

Referring to FIG. 23, the operation of the conventional ultrasonic image diagnosis apparatus involves ultrasonic transmission/reception and image synthesis processes similar to the configuration sequence of the conventional ultrasonic image diagnosis apparatus as shown in FIG. 21. The operational flow diagram of FIG. 23 forms a closed loop to repeatedly synthesize new image frames.

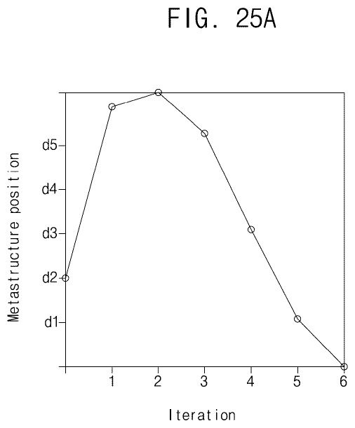

FIG. 24 is a flow chart for explaining an ultrasonic image diagnosis method using the apparatus of FIG. 22.

Alternatively, referring to FIG. 24, unlike the operation of the conventional ultrasonic image diagnosis apparatus as described with reference to FIG. 23, in the operation of the ultrasonic image diagnosis apparatus according to the present example embodiment, the position of the metastructure may be repeatedly controlled by evaluating the effect of the metastructure on the image, in addition to reducing the ultrasonic reflectivity of the multilayer barrier. At the same time, it has an operation method that changes the ultrasonic transmission waveform and searches for an ultrasonic transmission waveform suitable for the current metastructure-barrier multilayer system.

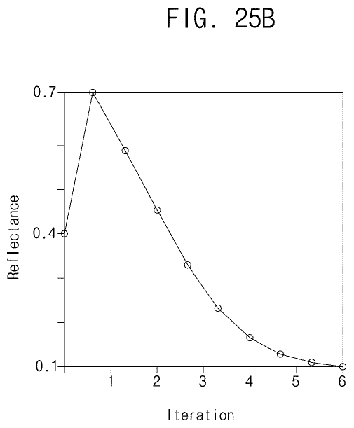

FIG. 25A and FIG. 25B are graphs showing an insertion position and reflectance change according to repetitive control of the metastructure, the method of FIG. 24.

That is, FIGS. 25A and 25B are graphs showing the insertion position and reflectance changes according to repetitive control of the metastructure in the method of operating the ultrasonic image diagnosis apparatus according to the present example embodiment.

When controlling the metastructure, the ultrasonic probe, and the ultrasonic image diagnosis apparatus, the thickness of the metastructure, the physical properties of the metastructure, the insertion location of the metastructure (matching distance), number of cycles of transmitted ultrasonic pulses, different time delay and amplitude weight (In FIG. 9B, the metastructure-multilayer barrier system is to correct for uneven ultrasonic transmittance by location) for each channel of transmitted and received ultrasonic waves during beamforming, etc. are important variables. In the end, FIG. 25A and FIG. 25B show control of the insertion position (matching distance) of the metastructure and change in reflectance, which may clearly show the impedance matching effect of the metastructure of the present example embodiment, among various important variables.

Specifically, FIG. 25A shows that the insertion position of the metastructure changes for each iteration, at this time, as shown in FIG. 25B, the reflectance of the multilayer system gradually decreases, and when it falls below a certain value, the movement of the metastructure stops. In addition, as shown in FIG. 25B, calculation of reflectance may be obtained by extracting the intensity of the signal reflected by the metastructure and the multilayer barrier among the received ultrasonic signals.

According to some exemplary embodiments of the present invention, the ultrasonic wave may be substantially 100% transparent across multilayer or non-uniform barriers.

In addition, when adjusting the matching distance of the metastructure or adjusting the physical properties such as the elastic modulus and pattern portion of the metastructure, the ultrasonic wave may be effectively transmitted while actively coping with the environmental conditions of the multilayer barrier.

In addition, the ultrasonic wave may be transmitted effectively despite individual differences in the physical properties of the skull as well as differences in physical properties by location.

In addition, the metastructure may be widely used. For example, the metastructure may be applied to medical ultrasonic imaging diagnosis and treatment in which the ultrasonic waves are transmitted into multilayered tissues containing substances with impedance differences such as bone tissue, soft tissue, gas, etc. In addition, the metastructure may be applied to various non-destructive testing industries that use the ultrasonic waves to penetrate multilayer physical barriers containing solid (metal, ceramic, plastic) or fluid materials (composite materials, insulation materials, rust or sludge in pipes, etc.).

Having described exemplary embodiments of the present invention, it is further noted that it is readily apparent to those of reasonable skill in the art that various modifications may be made without departing from the spirit and scope of the invention which is defined by the metes and bounds of the appended claims.

1. A metastructure for impedance matching,
wherein the metastructure is arranged to be spaced apart from a surface of a multilayer barrier by a predetermined matching distance toward an ultrasonic transducer, in an intermediate material provided between the ultrasonic transducer and the multilayer barrier,
wherein the metastructure is configured to minimize a difference between a surface impedance at the matching distance and a characteristic impedance of the intermediate material.

2. The metastructure of claim 1, wherein the metastructure has an anti-resonance mode in which magnitudes of positive and negative vibrations in a natural vibration mode are substantially equal.

3. The metastructure of claim 1, comprising:
a first material layer having a first elastic modulus and a first density; and
a second material layer having a second elastic modulus and a second density,
wherein the first and second material layers are stacked in an ultrasonic transmission direction,
wherein the first and second moduli are different from each other, or the first and second density are different from each other.

4. The metastructure of claim 3, wherein the second material layer is disposed on front and back sides of the first material layer with respect to the ultrasonic transmission direction.

5. The metastructure of claim 4, wherein the second material layer comprises a pattern unit having first and second areas, the first area exposes the first material layer to outside, and the second area covers the first material layer without exposing the first material layer.

6. The metastructure of claim 1, wherein the metastructure is a plural, and the plurality of the metastructures is arranged to tile each other in a direction crossing an ultrasonic transmission direction.

7. The metastructure of claim 1, comprising:
a first metastructure spaced apart by a first matching distance from the surface of the multilayer barrier; and
a second metastructure space apart by a second matching distance different from the first matching distance from the surface of the multilayer barrier.

8. A metastructure for impedance matching,
wherein the metastructure is arranged to be spaced apart from a surface of a multilayer barrier by a predetermined matching distance toward an ultrasonic transducer, in an intermediate material provided between the ultrasonic transducer and the multilayer barrier,
wherein the metastructure is configured to minimize a reflection coefficient of a system transfer matrix which consisting of a product of a transfer matrix expressed as a characteristic impedance and a phase change of each of the metastructure disposed at the matching distance, the intermediate material and the multilayer barrier.

9. The metastructure of claim 8, wherein the reflection coefficient of a system transfer matrix is minimized by the matching distance or a thickness of the metastructure.

10. The metastructure of claim 8, wherein the metastructure has an impedance twice or more than an impedance of the intermediate material or an impedance substantially the same as the characteristic impedance of the multilayer barrier, to expand a range of change in the system transfer matrix by intensifying multiple internal reflections of an ultrasonic wave propagating between the metastructure and the multilayer barrier.

11. A metastructure for impedance matching, the metastructure being disposed within an intermediate material provided between an ultrasonic transducer and a barrier, the metastructure comprising:
a first metastructure spaced apart by a first matching distance from a first surface area of the barrier toward the transducer, and configured to minimize a difference between a first surface impedance at the first matching distance and a characteristic impedance of the intermediate material; and
a second metastructure spaced apart by a second matching distance from a second surface area of the barrier adjacent to the first surface area toward the transducer, and configured to minimize a difference between a second surface impedance at the second matching distance and a characteristic impedance of the intermediate material.

12. The metastructure of claim 11, wherein the first metastructure has a first effective radiation area corresponding to the first surface area and the second metastructure has a second effective radiation area corresponding to the second surface area,
wherein each of the first and second effective radiation areas has a width smaller than ½ wavelength (λ).

13. A metastructure for impedance matching, the metastructure being disposed within an intermediate material provided between an ultrasonic transducer and a barrier, the metastructure comprising:
a first metastructure spaced apart by a first matching distance from a first surface area of the barrier toward the transducer, and configured to minimize a reflection coefficient of a first system transfer matrix which consisting of a product of a first transfer matrix expressed as a characteristic impedance and a phase change of each of the first metastructure disposed at the first matching distance, the intermediate material and the barrier; and
a second metastructure spaced apart by a second matching distance from a second surface area of the barrier adjacent to the first surface area toward the transducer, and configured to minimize a reflection coefficient of a second system transfer matrix which consisting of a product of a second transfer matrix expressed as a characteristic impedance and a phase change of each of the second metastructure disposed at the second matching distance, the intermediate material and the barrier.

14. The metastructure of claim 13, comprising:
a 1-1 metastructure disposed from the first surface area by a 1-1 matching distance; and
a 1-2 metastructure disposed from the first surface area by a 1-2 matching distance different from the 1-1 matching distance.

15. The metastructure of claim 13, wherein the first metastructure has an impedance twice or more than an impedance of the intermediate material or an impedance substantially the same as the characteristic impedance of the barrier, to expand a range of change in the first system transfer matrix by intensifying multiple internal reflections of an ultrasonic wave propagating between the first metastructure and the barrier.

16. An ultrasonic probe comprising:
the metastructure according to claim 1; and
an ultrasonic transducer configured to provide an ultrasonic wave to the metastructure.

17. The ultrasonic probe of claim 16, wherein the metastructure moves in an ultrasonic transmission direction with respect to the ultrasonic transducer.

18. An ultrasonic probe comprising:
the metastructure according to claim 11; and
an ultrasonic transducer configured to provide an ultrasonic wave to the metastructure.

19. The ultrasonic probe of claim 18, wherein the first metastructure or the second metastructure moves in an ultrasonic transmission direction with respect to the ultrasonic transducer.

20. An ultrasonic image diagnostic apparatus comprising:
the metastructure according to claim 1;
an ultrasonic transducer configured to provide an ultrasonic wave to the metastructure;
a signal processing unit configured to process a signal received in the ultrasonic transducer, to generate an ultrasonic image; and
a metastructure control unit configured to control at least one of physical properties, thickness and matching distance of the metastructure, based on the ultrasonic image generated in the signal processing unit.
